(19) United States
(12) Patent Application Publication
(10) Pub. No.: US 2022/0401214 A1
(43) Pub. Date: Dec. 22, 2022

(54) PROSTHETIC CARDIAC VALVE DELIVERY DEVICES, SYSTEMS, AND METHODS

(71) Applicant: SHIFAMED HOLDINGS, LLC, Campbell, CA (US)

(72) Inventor: Tom SAUL, Moss Beach, CA (US)

(21) Appl. No.: 17/773,193

(22) PCT Filed: Oct. 30, 2020
(86) PCT No.: PCT/US2020/058413

§ 371 Date: Apr. 29, 2022

Related U.S. Application Data
(60) Provisional application No. 62/927,922, filed on Oct. 30, 2019.

(51) Int. Cl. A61F 2/24

(52) U.S. Cl. CPC A61F 2/2418; A61F 2/2457; A61F 2/2466; A61F 2250/001

(57) ABSTRACT

A device and system for use with medical devices, such as catheter devices or systems. The device or system comprises an anchor for securing to tissue. The anchor comprises a series of segments that allow the anchor to be actuated from a delivery configuration to a deployed configuration. The anchor may include a tie band and a free end. In some examples, the device or system is used in treating a diseased native valve in a patient. The anchor may part of a delivery device to implant a prosthetic valve. Subsequent to delivery, the components of the delivery device are actuated to secure the prosthetic valve within the diseased valve.

### CROSS REFERENCE TO RELATED APPLICATIONS

This application claims priority to U.S. Provisional Application No. 62/927,922, filed on Oct. 30, 2019, entitled “PROSTHETIC CARDIAC VALVE DELIVERY DEVICES. SYSTEMS, AND METHODS”, the entirety of which is incorporated herein by reference for all purposes.

This application may be related to International Application No. PCT/US2019/055049, filed Oct. 7, 2019, entitled “PROSTHETIC CARDIAC VALVE DEVICES, SYSTEMS. AND METHODS”; International Application No. PCT/US2019/057082, filed Oct. 18, 2019, entitled “ADJUSTABLE MEDICAL DEVICE”; International Application No. PCT/US2019/068088, filed Dec. 20, 2019, entitled “PROSTHETIC CARDIAC VALVE DEVICES, SYSTEMS, AND METHODS”; International Application No. PCT/US2020/023671, filed Mar. 19, 2020, entitled “PROSTHETIC CARDIAC VALVE DEVICES, SYSTEMS, AND METHODS”; and International Application No. PCT/US2020/027744, filed Apr. 10, 2020, entitled “MINIMAL FRAME PROSTHETIC CARDIAC VALVE DELIVERY DEVICES, SYSTEMS, AND METHODS”; the entireties of which are incorporated herein by reference for all purposes.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Devices for use with medical devices and systems, such as catheter devices and systems. In some examples, the devices are used in delivering prosthetic cardiac valves, such as prosthetic mitral valves.

### BACKGROUND

Blood flow between heart chambers is regulated by native valves—the mitral valve, the aortic valve, the pulmonary valve, and the tricuspid valve. Each of these valves are passive one-way valves which open and close in response to differential pressures. Patients with valvular disease have abnormal anatomy and/or function of at least one valve. For example, a valve may suffer from insufficiency, also referred to as regurgitation, when the valve does not fully close and allows blood to flow retrograde. Valve stenosis can cause a valve to fail to open properly. Other diseases may also lead to dysfunction of the valves. While medications may be used to treat the disease, in many cases the defective valve may need to be repaired or replaced at some point during the patient's lifetime. Existing valves and surgical repair and/or replacement procedures may have increased risks, limited lifespans, and/or are highly invasive. Some less-invasive transcatheter options are available, however these generally are limited to aortic valve procedures, are limited in their patient-to-patient flexibility, and often take longer than desired to implant. Currently available procedures often require the placement of more than one component—for example, a prosthetic valve and a mechanism to anchor it to the native anatomy. Such procedures generally utilize multiple delivery catheters to carry the various components and delivery of each component separately to the valve, which can be time-consuming (particularly if components are delivered sequential), complicated, and/or dangerous. It would therefore be desirable to provide a valve assembly for valvular replacement and repair wherein the components are controlled and contained within a single delivery device.

### SUMMARY

Described herein is a less invasive procedure for repair and replacement of heart valves, including the mitral valve, quicker surgical methods, and/or prosthetic valves that can accommodate a variety of individual patients. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

The present disclosure generally relates to treating a diseased native valve in a patient and more particularly relates to prosthetic heart valves with self-assembling anchor elements.

Disclosed herein is a system for treating a diseased native valve in a patient, the system comprising: a valve prosthesis, the valve prosthesis comprising a frame structure and an anchor; the frame structure having an unexpanded configuration and an expanded configuration; the anchor comprising a series of segments operably coupled to one another by a tie band and having a free end, wherein the frame structure is configured to be actuated from the unexpanded configuration to the expanded configuration adjacent a native valve in a patient, wherein the anchor is configured to be deployed from an anchor deployment component of a delivery device; and wherein the anchor is configured to secure the valve prosthesis to the native valve when the valve prosthesis is deployed into the expanded configuration adjacent the native valve. The anchor deployment component can comprise an anchor delivery gear. The series of connected segments can comprise a gear interface located opposite to a tie relief cut, wherein the gear interface is configured to attach to the anchor delivery gear. The tie band can be a wire and the segments can be configured to hold the wire wherein the segments can further comprise a tie band wire relief on an inner face of the segments to allow the wire to bulge when the anchor is in a delivery configuration. A side edge of each of the series of connected segments can be slanted relative to a central axis of the anchor. Each of the series of connected segments can comprise a drive pin configured to be attached to a delivery device when the anchor is in a deployed configuration. The connected segments can each comprise a tie band slot, tie band retaining pin, and tie band retaining tabs on an inner edge of each segment to hold the tie band in place. The tie band can comprise tie hand retaining pin constraints and fold relief slots to control movement of the anchor from a delivery gear configuration to a deployed configuration. The fold relief slots can be slanted. The fold relief slots can be normal to a spine of the anchor. The connected segments can be connected by knuckle elements on side edges of each segment configured to be complementary to a knuckle element on a side edge of an adjacent segment. The knuckles can comprise a hinge pin configured to hold complementary knuckle elements together. An edge of each segment, located adjacent to a proximal segment, can be slanted in order to assemble the anchor from the delivery configuration to the deployed configuration. The anchor can be configured to be advanced from an atrial side of the native valve into a ventricle of the heart and to be fully deployed into the ventricle in a deployed configuration. The delivery device can comprise a delivery gear comprising a delivery pin guide on the surface of the delivery gear. The anchor can be released from a delivery configuration around the delivery gear to a delivered configuration. A drive pin of each segment can be attached to the anchor deployment component in the delivery configuration. The delivery gear can comprise an outer shaft, an inner shaft within the outer shaft, and an anchor delivery gear within the inner shaft, wherein the anchor delivery gear comprises a deployment drive. The delivery device can further comprise a side port in the inner shaft of the delivery device from which the anchor is deployed. The anchor can comprise a delivery configuration and a deployed configuration. The anchor can comprise a spiral shape in the delivery configuration. The anchor can be configured to be actuated from the delivery configuration to the deployed configuration by being released from an anchor delivery drive. The anchor delivery drive can comprise a delivery pin guide and each segment of the anchor can comprise a drive pin configured to attach to the delivery pin guide. The anchor delivery drive can comprise a series of teeth and each segment of the anchor can comprise a gear interface configured to attach to the teeth of the anchor delivery drive. The anchor can comprise a spiral shape in the deployed configuration. The anchor can comprise a circular shape in the deployed configuration. The anchor can be configured to be actuated from the delivery configuration to the deployed configuration adjacent the native valve. The anchor can be configured to be deployed adjacent the native valve. The anchor can comprise a super-elastic material. The anchor can comprise nitinol. The free end of the anchor can comprise an atraumatic tip. The free end of the anchor can comprise a ball tip. The free end of the anchor can be configured for piercing tissue. The free end of the anchor can be bent distally. The free end of the anchor can be bent proximally. The free end of the anchor can be disposed radially outwards from the support structure. The connected segments can comprise a lumen and a wire disposed within the lumen. The frame structure can be configured to expand within the native valve of the patient. The unexpanded configuration can be sized and dimensioned for percutaneous insertion and the expanded configuration can be sized and dimensioned for implantation in the native valve of the patient. The valve prosthesis can comprise a first and second opposite ends, the first end being configured to extend above a native valve and the second end being configured to extend below the native valve and the second end being configured to extend below the native valve when the valve prosthesis is anchored to the native valve. The valve prosthesis can be configured to sit below the native valve when the frame structure is anchored to the native valve. The valve segment within the valve prosthesis can comprise a biocompatible one-way valve. At least a portion of the valve segment can be positioned within at least a portion of the valve prosthesis. The valve segment can comprise at least one leaflet having an inner layer and an outer layer wherein the frame structure is attached to the outer layer at one or more ends of the valve prosthesis. The valve segment can comprise a plurality of leaflets.

Disclosed herein is a system for treating a diseased native valve in a patient, the system comprising a valve prosthesis, the valve prosthesis comprising a frame structure and an anchor; the frame structure having an unexpanded configuration and an expanded configuration; the anchor comprising a series of segments laser cut into a hollow band and having a free end, wherein the frame structure is configured to be actuated from the unexpanded configuration to the expanded configuration adjacent a native valve in a patient, wherein the anchor is configured to be deployed from an anchor deployment component of a delivery device; and wherein the anchor is configured to secure the valve prosthesis to the native valve when the valve prosthesis is deployed into the expanded configuration adjacent the native valve; and wherein the anchor is configured to have a low compressive stiffness and low expansive stiffness in the delivery configuration and a high expansion stiffness after transition to the deployed configuration.

Disclosed herein is a device for treating a diseased valve in a patient, the device comprising: a valve prosthesis comprising a frame structure and an anchor, wherein the anchor comprises a series of segments operably coupled to one another by a tie band and having a free end, wherein the anchor has a delivery configuration further comprising an anchor delivery gear within a delivery device and a deployed configuration, wherein the anchor is configured to secure the valve prosthesis to the diseased valve. Each of the connected segments can comprise a gear interface and a tie relief cut. The connected segments can comprise and spine and a tie band. The tie band of the connected segments can be a wire and each segment including a tie band wire slot. An edge of each connected segment can be slanted. Each connected segment can comprise a drive pin. Each connected segment can comprise a tie band wire relief. The connected segments can comprise a tie band slot and tie band retaining tabs. The connected segments can be connected by attachment to a tie band comprising fold relief slots and tie band retaining pin constraints. The fold relief slots can be slanted. The fold relief slots can be straight. The connected segments can be connected by knuckles. The knuckles can comprise hinge pins. The knuckles can be connected by a wire. An edge of each segment can be slanted. The anchor can be deployed from an anchor deployment component of a delivery device. The anchor can be configured to secure the valve prosthesis to the native valve when the valve prosthesis is deployed into the expanded configuration adjacent the native valve.

Disclosed herein is a device for treating a disease valve in a patient, the device comprising: a valve prothesis comprising a frame structure and an anchor, wherein the anchor comprises a series of segments operably coupled to one another by a tie band and having a free end, wherein the anchor has a delivery configuration further comprising an anchor delivery gear within a delivery device and a deployed configuration, wherein the anchor is configured to secure the valve prosthesis to the diseased valve. The free end can comprise an atraumatic tip. The free end can comprise a ball tip. Each of the connected segments can comprise a gear interface and a tie relief cut. The connected segments can comprise a spine and a tie band. The tie band of the connected segments is a wire, and each segment comprises a tie band wire slot. An edge of each connected segment can each be slanted. The connected segments can comprise a drive pin. The connected segments can each comprise a tie band wire relief. The connected segments each can comprise a tie band slot and tie band retaining tabs. The connected segments can be connected by attachment to a tie band comprising fold relief slots and tie band retaining pin constraints. The fold relief slots can be slanted. The fold relief slots can be perpendicular to and edge of the tie band. The connected segments can be connected by knuckles. The knuckles can comprise hinge pins. The knuckles can be connected by a wire. An edge of each segment can be slanted. The anchor can be deployed from the inner shaft through a port located on the side of the inner shaft or through a distal end of the inner shaft. The anchor can be deployed by a retraction of an outer shaft followed by a series of rotations of an anchor drive shaft followed by an advancement of the outer shaft, followed by an exposure of a deployment drive. The frame structure can be deployed after the anchor is deployed. Placement of the valve prosthesis can be facilitated by an opening and a closing of the valve during cardiac cycle. The frame prosthesis can be released from a balloon when in the expanded position. The anchor can be anchored to one or more native leaflets and/or one or more native chordae tendineae of the left ventricle.

Disclosed herein is a method for treating a diseased native valve in a patient, the method comprising: deploying an anchor with a delivery device from a delivery configuration to a deployed configuration; wherein the anchor comprises segments connected in series by a tie band; wherein deploying the anchor includes wrapping the anchor around native leaflets, native chordae tendineae, or the native leaflets and native chordae tendineae adjacent to the native valve; expanding a frame structure within the native valve adjacent the deployed anchor from an unexpanded configuration to an expanded configuration to secure the anchor to the native leaflets, native chordae tendineae, or the native leaflets and the native chordae tendineae; and retracting the delivery device from the native valve. Deploying the anchor from the delivery device can further comprise releasing the segments from an anchor delivery gear within the delivery device. Deploying the anchor from the delivery device can further comprise releasing a pin on each of the segments from a delivery pin guide of the anchor delivery gear. Securing the anchor can further comprise rotating the anchor around the native leaflets, native chordae tendineae, or the native leaflets and the native chordae tendineae. Deploying the anchor from the delivery device can further comprise releasing a gear interface on each of the segments from a tooth of the anchor delivery gear. The segments can be connected to an anchor delivery gear in the delivery configuration. Securing the anchor can occur simultaneously with the deployment of the anchor, further comprising rotating the anchor around the native leaflets, native chordae tendineae, or the native leaflets and the native chordae tendineae. Deployment of the anchor can occur in the left ventricle.

Disclosed herein is a device for treating a disease valve in a patient, the device comprising: a valve prothesis comprising a frame structure and an anchor, wherein the anchor comprises a series of segments operably coupled to one another and having a free end, wherein the anchor has a delivery configuration further comprising a helical configuration around an anchor delivery gear within a delivery device and a deployed configuration further comprising a circular configuration, wherein the anchor is configured to secure the valve prosthesis to the diseased valve; wherein the anchor is transitioned from the delivery configuration to the deployed configuration by rotating the delivery gear.

Disclosed herein is a device for treating a disease valve in a patient, the device comprising: a valve prothesis comprising a frame structure and an anchor, wherein the anchor comprises a series of segments operably coupled to one another and having a free end, wherein the anchor has a delivery configuration and a deployed configuration, wherein the anchor is configured to secure the valve prosthesis to the diseased valve; wherein the anchor is configured to have a low compressive stiffness and low expansive stiffness in the delivery configuration and a high expansion stiffness after transition to the deployed configuration.

Disclosed herein is a device for treating a patient, the device comprising: a component comprising a series of segments operably coupled to one another and having a free end, wherein the component has a helically shaped radially collapsed delivery configuration and a radially expanded deployed configuration, wherein the segments are configured to minimize expansion of the component when the component is in the radially collapsed delivery configuration, further wherein the device is configured to have a low compressive and expansive stiffness in the radially collapsed delivery configuration and a high expansion stiffness when in the radially expanded deployed configuration. Each of the segments can comprise a gear interface and a tie relief cut. The component can include a spine and a tie band. The tie band can be a wire and each segment can comprise a tie band wire slot. At least one edge of the segments can be slanted. The segments can each comprise a drive pin. Each of the segments can comprise a tie band wire relief. Each of the segments can comprise a tie band slot and tie band retaining tabs. The segments can be connected by attachment to a tie band comprising fold relief slots and tie band retaining pin constraints. The fold relief slots can be slanted. The fold relief slots can be perpendicular to an edge of the tie band. The segments can be connected by knuckles. The knuckles can comprise hinge pins. The knuckles can be connected by a wire. At least one edge of the segments can be slanted. The anchor can be configured to be in the radially collapsed delivery configuration when within a delivery device. The delivery device can be a delivery catheter. The anchor can be configured to be deployed from an inner shaft of the delivery device through a port located on a side of the inner shaft or out a distal end of the inner shaft. The anchor can be configured to be deployed by a retraction of an outer shaft followed by a series of rotations of an anchor drive shaft, followed by an advancement of the outer shaft, and followed by an exposure of a deployment drive. The device further can comprise a valve prothesis comprising a frame structure, wherein the component is configured to secure the valve prosthesis to a diseased valve. The frame structure can be deployed after the component is deployed. Placement of the valve prosthesis can be facilitated by an opening and a closing of the diseased valve during a cardiac cycle. The valve prosthesis can be released from a balloon when the valve prosthesis is expanded to an expanded position. The component can be configured to anchor to one or more native leaflets, one or more native chordae tendineae, or to one or more native leaflets and one or more native chordae tendineae associated with the diseased valve. The free end can be configured to extend radially outward when being deployed. The free end can comprise an atraumatic tip. The free end can comprise a ball tip. The component can include a locking mechanism configured to lock two ends of the component together when in the radially expanded deployed configuration. A distal end of the component can comprise a key configured to slide into a complementary lock located on a band of the component. The component can have a flat shape when in the radially expanded deployed configuration. The component can be wrapped around a delivery gear when in the radially collapsed delivery configuration. The component can be part of a catheter system, wherein the catheter system is one or more of: an imaging catheter system, a diagnostic catheter system, a delivery catheter system, a catheter-based therapeutic device system, and a robotic surgery catheter system. The component can be configured to secure a catheter or a device as part of a catheter-based system to a location in the human anatomy. The series of segments can be operatively coupled to one another by a tie band. The tie band can be a wire. The wire can be a spring. The component can be configured to coil around a delivery gear when in the radially collapsed delivery configuration. Each of the series of segments can have angled edges in accordance with a delivery pitch angle for coiling the anchor around the delivery gear such that the component has a flat shape when in the radially expanded deployed configuration. The delivery pitch angle can range from 5 degrees and 85 degrees. Each of the series of segments can have angled edges that allow the component to take on the helical shape when in the radially collapsed delivery configuration. The component can include a tie band that has pin portions positioned within knuckles of the segments. The component can include a spring that maintains the component in the radially expanded deployed configuration. The spring can be a band or wire that is adjacent to the segments or runs through the segments. The segments can be pseudo units connected by a material at an inner radius of the anchor. The material at the inner radius of the anchor can correspond to a tie band that applies a radial expansion force to maintain the anchor in the radially expanded deployed configuration. The segments can be units that are single entities coupled together by a coupling structure. The coupling structure can be a band or wire that applies a radial expansion force to maintain the anchor in the radially expanded deployed configuration.

As described herein, the anchors can include segments that correspond to longitudinal sections of an anchor and that maintain their shape as the anchor transitions between a collapsed delivery configuration and an expanded deployed configuration. The segments can include pseudo units and units (also referred to has links), as described herein.

These and other embodiments are described in further detail in the following description related to the appended drawing figures.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIGS. 1A-1C show a side cross-sectional view, a top view, and a bottom view, respectively, of a heart having a diseased mitral valve which may be treated using the devices, systems, and methods described herein, in accordance with many embodiments.

FIGS. 2A-2B show a side cross-sectional view and a top view, respectively, of a heart after repair of a diseased mitral valve with a valve prosthesis, in accordance with many embodiments.

FIGS. 3A-3F show side cross-sectional views of the deployment of an anchor from a delivery device, in accordance with many embodiments.

FIGS. 4A-4D show perspective views of deployment of an anchor from a flat screw embodiment of the delivery device, in accordance with many embodiments

FIG. 5A shows a top view of a laser-cut anchor in a deployed configuration, in accordance with many embodiments.

FIG. 5B shows a top view of the laser-cut anchor of FIG. 5A highlighting the diameter of the delivery configuration, in accordance with many embodiments.

FIG. 6 shows a perspective view of the laser-cut anchor of FIG. 5A in the deployed configuration following deployment from a delivery gear, in accordance with many embodiments.

FIG. 7A shows a perspective view of the laser-cut anchor of FIG. 5A in a delivery configuration around a delivery gear, the delivery gear comprising a delivery tooth configured to hold the anchor in the delivery configuration, in accordance with many embodiments.

FIG. 7B shows a side view of the laser-cut anchor of FIG. 5A in the deployed configuration, in accordance with many embodiments.

FIG. 8 shows a close perspective view of the laser-cut anchor of FIG. 5A in the deployed configuration, highlighting the gear interface of the laser cut anchor, in accordance with many embodiments.

FIG. 9 shows a view of a laser-cut anchor in a deployed configuration, in accordance with many embodiments.

FIG. 10 shows a close view of the laser-cut anchor of FIG. 9, the anchor comprising a tie band flanked by cuts that can serve as tie relief cuts and gear interfaces, in accordance with many embodiments.

FIG. 11 shows a top view of a multi-link anchor in a (nearly) deployed configuration, in accordance with many embodiments.

FIG. 12 shows a perspective view of the multi-link anchor of FIG. 11 highlighting the tie band wire slots of the anchor, in accordance with many embodiments.

FIG. 13 shows a close side view of the multi-link anchor of FIG. 11 highlighting the angled edges of each unit configured to facilitate wrapping into a delivery configuration, in accordance with many embodiments.

FIG. 14 shows a close perspective view of the multi-link anchor of FIG. 12 showing the tie hand wire slot of the anchor, in accordance with many embodiments.

FIG. 15 shows a top view of a multi-link anchor comprising unit drive pins on each unit of the anchor, the unit drive pins being configured to uncouple to a delivery drive as the anchor is deployed from a delivery configuration around the delivery drive to a deployed configuration, in accordance with many embodiments.

FIG. 16 shows a perspective view of the anchor of FIG. 15 highlighting the placement of the drive pins in a delivery pin guide of the delivery drive, in accordance with many embodiments.

FIG. 17 shows a side view of the anchor of FIG. 15 with pins showing the deployment of the anchor from the delivery configuration around the delivery drive to the deployed configuration, in accordance with many embodiments.

FIG. 18 shows a close perspective view of the anchor of FIG. 15, the anchor comprising tie band wire relief present on each unit of the anchor, in accordance with many embodiments.

FIGS. 19A-19C show close views of a unit of an anchor with pins, each unit comprising a tie band retaining pin and tie band retaining tabs, in accordance with many embodiments. FIG. 19A shows a top perspective view. FIG. 19B shows a side plan view. FIG. 19C shows a side view.

FIGS. 20A-20B show close perspective and side views, respectively, of a tie band comprising tie band retaining pin constraints and fold relief slots patterned along the length of the tie band, in accordance with many embodiments.

FIGS. 21A-21B shows top and perspective views, respectively, of an anchor comprising pins, the anchor being deployed from a delivery configuration disposed around a delivery drive to a deployed configuration, in accordance with many embodiments.

FIGS. 22A-22C show close views of a unit of the anchor of FIGS. 21A-21B, each unit comprising a knuckle configured to couple to a knuckle of an adjacent unit with a pin therebetween, in accordance with many embodiments. FIG. 22A shows a top perspective view. FIG. 22B shows a top view. FIG. 22C shows a side view.

FIG. 23 shows a close perspective view of the anchor of FIGS. 21A-21B with hinge pins located between units and holding each unit together at their knuckles, in accordance with many embodiments.

FIG. 24 shows a side view of an anchor of FIGS. 21A-21B being deployed from the delivery configuration around the delivery drive to the deployed configuration, in accordance with many embodiments.

FIGS. 25A-25F show various views of an example unit for an anchor illustrating various geometric aspects and dimensions of the unit: FIG. 25A shows a side view illustrating various geometric aspects; FIG. 25B shows a perspective view; FIG. 25C shows a perspective view of the unit illustrating various dimensions; FIG. 25D shows a side view; FIG. 25E shows another side view; and FIG. 25F shows a view of the inner side of the unit.

FIGS. 26A-26D show various views of an anchor in a partially deployed state and formed using multiple unit s of FIGS. 25A-25F: FIG. 26A shows an aerial view of the anchor; FIG. 26B shows a perspective view of the anchor; FIG. 26C shows a side view of the anchor; and FIG. 26D shows another side view of the anchor.

FIG. 27 shows a perspective view of an anchor having a tie band that includes pin portions.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure is described in relation to deployment of systems, devices, or methods for treatment of a diseased native valve of the heart, for example a mitral valve. However, one of skill in the art will appreciate that this is not intended to be limiting and the devices and methods disclosed herein may be used in other anatomical areas and in other surgical procedures.

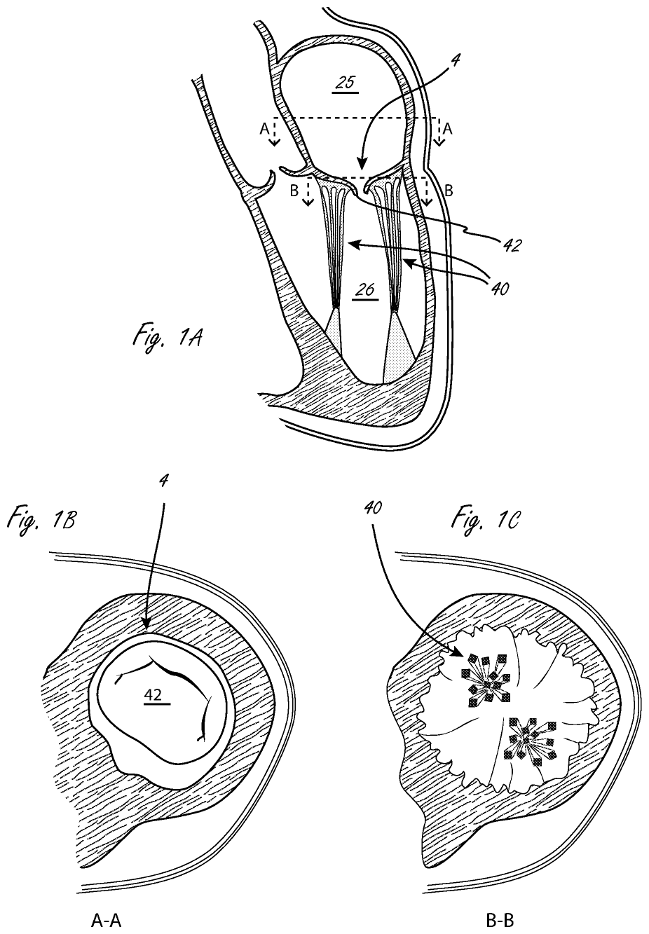

FIG. 1A shows a cross section view of a heart having a diseased mitral valve 4 which may be treated using the devices, systems, and methods described herein. The mitral valve 4 sits between the left atrium 25 and the left ventricle 26 and, when functioning properly, allows blood to flow from the left atrium 25 to the left ventricle 26 while preventing backflow or regurgitation in the reverse direction. As shown in FIG. 1A, the native valve leaflets 42 of the diseased mitral valve 4 do not fully prolapse and the patient experiences regurgitation. The native chordae tendineae 40 of the heart 1 are shown. FIG. 1B shows a cross-sectional view of the heart 2 taken along line A-A, shown in FIG. 1A, which shows the native valve leaflets 42 of the mitral valve 4 from the viewpoint of the left atrium 25. FIG. 1C shows a cross-sectional view of the heart 2 taken along line B-B, shown in FIG. 1A, which shows the chordae tendineae 40 of the left ventricle 26.

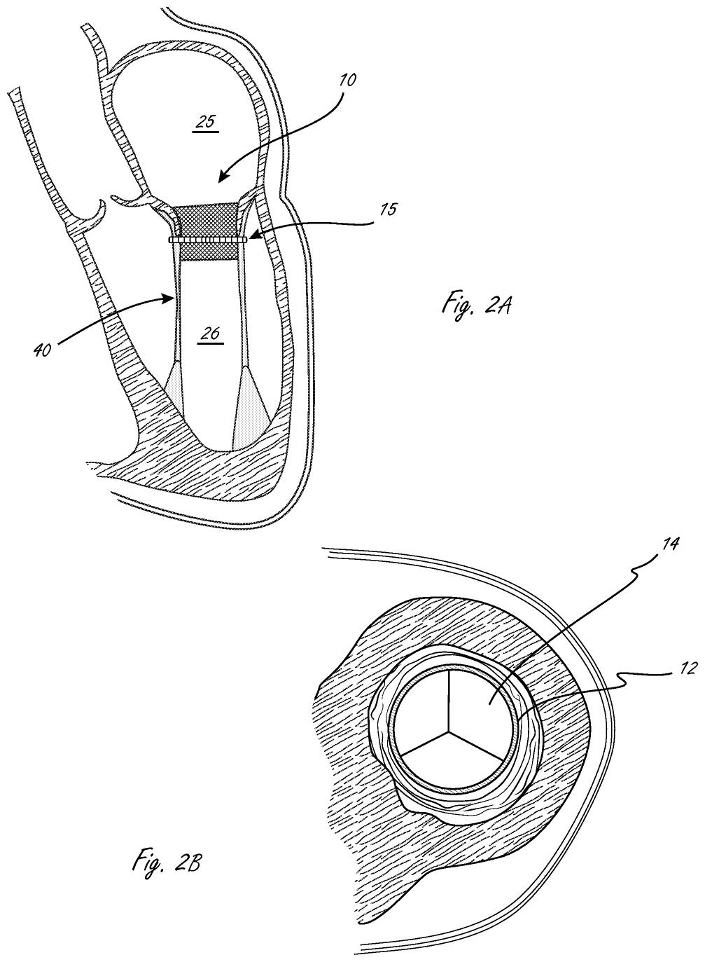

FIG. 2A is a section view of a diseased valve comprising a left atrium 25 and a left ventricle 26 with a valve prosthesis 10 comprising a frame structure 12 and an anchor 15 (also referred to herein as a component) disposed therein. The anchor encircles native chordae tendineae 40 of the native valve and captures leaflet tissue between the anchor and the valve. FIG. 2B is a top view of the valve prosthesis 10 showing the leaflets 14 and the frame structure 12.

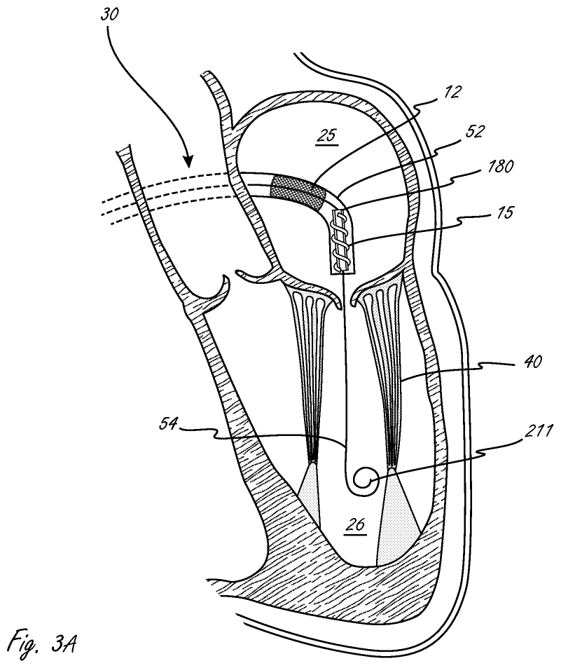

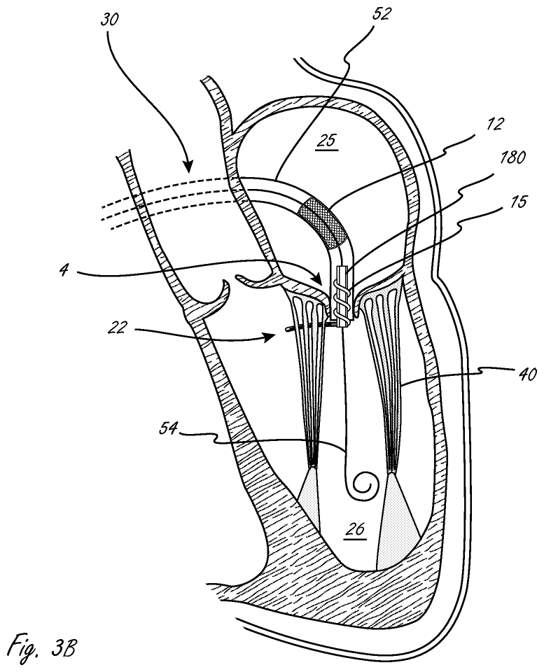

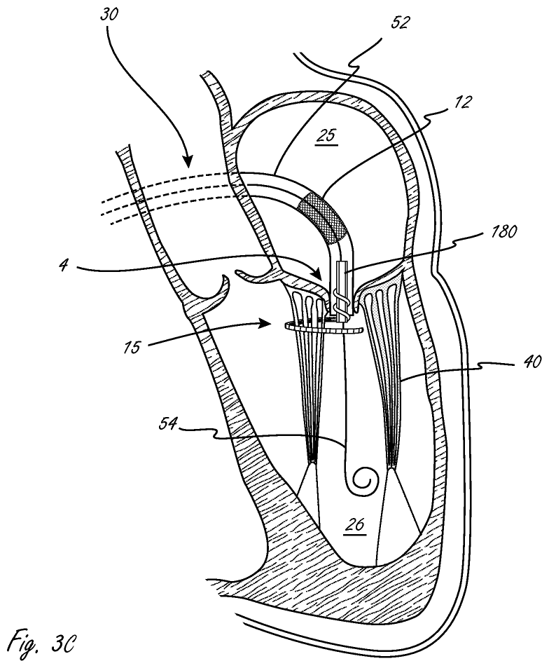

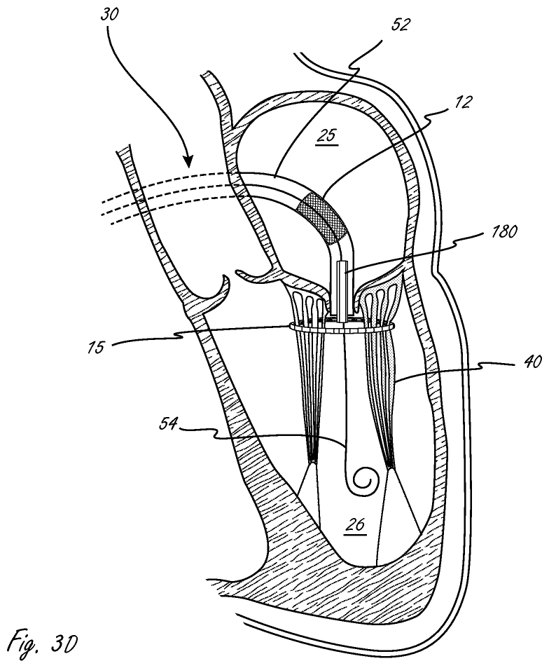

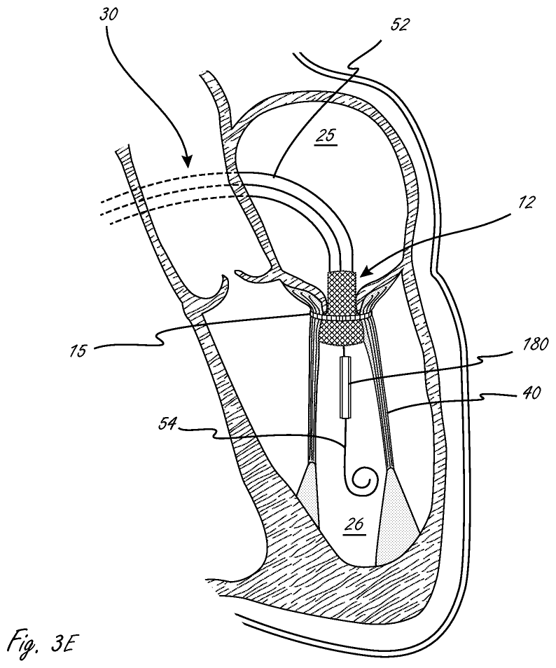

FIGS. 3A-3F show sequential views of a method of implanting a valve prosthesis 10 using a delivery device 30. The valve prosthesis 10 may be similar to any of the valve prostheses described herein or understood by one of ordinary skill in the art from the description herein. For example, valve prosthesis 10 may comprise a frame structure 12 and anchor 15 as described herein. The delivery device 30 may comprise an inner shaft 52 as described herein. The delivery device 30 may optionally comprise an outer shaft, a guidewire 54, a proximal pusher, in any combination thereof as desired by one of ordinary skill in the art. The proximal pusher may comprise a flexible advancement member housed and advanceable within the inner shaft 52, for example as described in 62/833,430, previously incorporated herein by reference for all purposes. Not all elements are labeled in each of FIGS. 3A-3F in order to make the illustrations less cluttered and easier to see.

While the methods, devices, and systems described herein are described in relation to a mitral valve replacement procedure, it will be understood by one of ordinary skill in the art that the methods, devices, and systems described herein may be applied to a variety of procedures or anatomical areas, for example other atrioventricular valves of the heart or the like.

A distal end of the delivery device 30 may be inserted into the left atrium 25 of the heart 2 via a transseptal puncture as described herein. For example, the guidewire 54 may be advanced into the left ventricle 26 through the left atrium 25 of the heart 2. The inner shaft 52 may be advanced distally into the left atrium 25 following the of the guidewire 54. In some embodiments, advancing the inner shaft 52 relative to the guidewire 54 may aid in deployment and/or placement of the valve prosthesis 10 as described herein. Both the guidewire 54 and the inner shaft 52 may be advanced distally into the left atrium 25 through the transseptal puncture.

FIG. 3A shows the delivery device 30 in an advanced position with the distal end of the guidewire 54 in the left ventricle 26 and the inner shaft 52 in the left atrium 25. The anchor 15 can comprise a delivery configuration around an anchor drive shaft 180. The anchor 15 and anchor drive shaft 180 may be located distal of the frame structure 12 of the valve prosthesis 10 inside the inner shaft 52. The anchor 15 can be maintained in the delivery configuration by radial constriction by the inner shaft 52.

The distal end of the delivery device 30 (for example, the distal end of the inner shaft 52 and/or the guidewire 54) may be steered such that the distal end of the delivery device 30 points toward the atrial side of the native valve 4. Such steering may occur prior to, during, or after deployment of at least a portion (for example, deployment of an anchor 15) of the valve prosthesis 10. In some embodiments, the distal end of the guidewire 54 may be steerable. Alternatively, or in combination, the inner shaft 52 may comprise a joint configured to change an angle of the distal portion of the inner shaft 52 relative to a proximal portion of the inner shaft 52. The inner shaft 52 may be steered by changing the angle of the distal portion of the inner shaft 52 relative to the proximal portion of the inner shaft 52. The angle of the joint may be changed passively or actively. In various embodiments, the angle may be selectively controlled by a proximal handle. For example, pull wires or other mechanisms may connect to the joint to controls on the handle.

FIGS. 3B-3D show the delivery of the anchor 15 from the delivery device 30. FIG. 3B shows the advancement of the inner shaft 52 to the diseased valve 4, where the anchor 15 is delivered into a ventricle side of the diseased valve 4. FIG. 3C shows the anchor 15 partially delivered to the native valve 4. FIG. 3D the anchor 15 in a deployed configuration around the native leaflets 42 and/or the native chordae tendineae 40 of the diseased valve 4.

As shown in FIGS. 3B-3C, the anchor 15 can be delivered from a distal end of the inner shaft 52. The anchor 15 can be released from a delivery configuration around the anchor drive shaft 180 by the advancement of the anchor drive shaft 180 up to and/or out of the distal end of the inner shaft 52. The anchor 15 may be progressively unwound from the anchor drive shaft 180 when at least a portion of the anchor drive shaft 180 is exposed at or from the distal end of the inner shaft 52. In some embodiments, the anchor 15 may be retained in the delivery configuration around the anchor drive shaft 180 due to radial constraint from the inner shaft 52. As the anchor drive shaft 180 is pushed out of the distal end of the inner shaft 52, the anchor 15 may be released from radial constraint and allowed to unwrap into the deployed configuration. Alternatively, or in combination, the anchor 15 may be deployed by rotation of the anchor drive shaft 180, which may facilitate unwinding of the anchor 15 as described herein. Deployment of the anchor 15 may occur entirely on the ventricular side of the native valve 4. In some embodiments, deployment of the anchor 15 may occur simultaneously with capture of the native leaflets 42 and/or the native chordae tendineae 40 as described herein. For example, as the anchor 15 unwinds into the deployed configuration, the deploying portion of the anchor 15 may wrap around the native leaflets 42 and/or the native chordae tendineae 40 as it changes between configurations. FIG. 3D shows the anchor 15 in the fully deployed configuration around the native leaflets 42 and/or the native chordae tendineae 40.

Alternatively, the anchor may be delivered from a lateral opening. The lateral opening may, for example, comprise a side port. Alternatively. or in combination, the lateral opening may comprise an opening which exposes the distal end of the anchor resultant of a retraction of an outer shaft. As described herein, at least a portion of the valve prosthesis 10 may be deployed from an undeployed (for example, compressed or unexpanded) configuration to an expanded configuration within the left ventricle 26. At least a portion of the anchor 15 may be deployed from a delivery and/or elongated configuration to a deployed configuration within the heart. For example, anchor 15, may be actuated from an elongated configuration to a deployed configuration within the left ventricle 26 as described herein. In some embodiments, the anchor 15 may be deployed from the inner shaft 52 by pushing the anchor 15 out of the side port of the inner shaft 52 (e.g., with a proximal pusher as described herein), releasing the anchor 15 from radial constraint by retracting the outer shaft 50, or the like as described herein. After the anchor 15 has been deployed from the delivery device 30, the valve prosthesis 10 comprising frame structure 12 may be at least partially deployed from the delivery device 30 (e.g., as shown in FIGS. 3E-3F) so as to place the frame structure 12 within the anchor 15. The valve prosthesis 10 comprising frame structure 12 may be deployed from the inner shaft 52 of the delivery device 30 in either the unexpanded configuration or the expanded configuration, depending on the location of deployment, as will be understood by one of ordinary skill in the art.

FIG. 3E shows the delivery of the frame structure 12 to the diseased valve 4. The anchor drive shaft 180 can be advanced along the guidewire 54, or the anchor drive shaft 180 and the guidewire 54 may be advanced together, distally out of the inner shaft 52. The frame structure 12 may be similarly advanced distally as the anchor drive shaft 180 is advanced. The frame structure 12 can be deployed into an expanded configuration within the diseased valve 4 as it is advanced out of the distal end of the inner shaft 52. Advancing the frame structure 12 out of the distal end of the inner shaft 52 may release the frame structure 12 from the radial constriction provided by the inner shaft 52 and allow it to expand to the expanded configuration within the diseased valve 4.

The frame structure 12 may be expanded within the native valve 4 from an unexpanded configuration to an expanded configuration. In some embodiments, at least a portion the frame structure 12 may be expanded within at least a portion of the deployed anchor 15 to anchor the frame structure 12 to the native valve 4. In some embodiments, the frame structure 12 may comprise an expandable stent. In some embodiments, the frame structure 12 of valve prosthesis 10 may be balloon-expandable. In some embodiments, the frame structure 12 of valve prosthesis 10 may be self-expandable. The delivery device 30 may comprise a proximal pusher which may be disposed within the valve prosthesis 10 in order to expand the valve prosthesis 10 as described herein. Alternatively, or in combination, the valve prosthesis 10 may be coupled to the guidewire 54 such that translation of the guidewire 54 translates the valve prosthesis 10 within the inner shaft 52.

The frame structure 12 may be partially expanded following translation out the distal end of the inner shaft 52, for example self-expanded after being partially pushed by a pusher or translated by the guidewire 54 out of a distal end of the inner shaft 52. The frame structure 12 may be deployed from a distal end of the delivery device 30 The frame structure 12 may be at least partially expanded towards the anchor 15 in order to capture the native leaflets 42 and/or the native chordae tendineae 40 therebetween. As the frame structure 12 continues to be expanded to a fully expanded state, for example by continued advancement of the frame structure 12, the native leaflets 42 and/or the native chordae tendineae 40 may be sandwiched between the anchor 15 and the frame structure 12. The frame structure 12 and anchor 15 may thus be anchored to the native leaflets 42 and/or the native chordae tendineae 40 as shown in FIG. 3F.

FIG. 3F shows the delivered configuration of the valve prosthesis 10 inside the native valve 4. The frame structure 12 is fully expanded within a diseased valve 4, locked in place by the anchor 15, with the native leaflets 42 and/or the native chordae tendineae 40 captured therebetween. Once the frame structure 12 has been fully expanded, the anchor drive shaft 180 can be retracted into the inner shaft 52, along with the guidewire 54, as the inner shaft 52 is removed from the left atrium 25 of the heart 2.

The distal end of the delivery device 30 and/or valve prosthesis 10 may be configured to be advanced from a first side of a native valve to a second side of the native valve. For example, the distal end of the delivery device 30 and/or valve prosthesis 10 may be advanced from a left atrial side of a mitral valve 4 to a left ventricular side of a mitral valve 4.

The valve prosthesis 10 may be secured to an anchor which has encircled enough of the leaflets and/or chordae to allow the anchor to reside just below the atrial side of the leaflets proximal the leaflet anulus Some chorea 40 may also be in communication with the valve prosthesis 10. As described further herein, the prosthesis 10 may be further anchored by expansion of the frame structure 12 within the native valve 4 and against the anchor 15.

Rotation of the anchor 15 may occur simultaneously with deployment of the anchor 15 (e.g., unwinding of the anchor 15 from an anchor drive shaft may effectively rotate the anchor 15 around the one or more structures with or without additional rotational motion being applied by the delivery device). The one or more structures may comprise one or more valve leaflets 42 and/or one or more chordae tendineae 40 (e.g., as shown in FIGS. 1A-1C). After the anchor 15 has been placed within the left ventricle 26 adjacent leaflets 42, the valve prosthesis 10 (e.g., the anchor 15 and, optionally, the frame structure 12) may be rotated to capture and anchor the native chordae 40 and/or native leaflets 42. The free end 22 of the anchor 15 may extend radially outward from the rest of the anchor 15 to facilitate capture of the native structures. The free end 22 of the anchor 15 may be rotated around one or more leaflets 42 and/or one or more of the chordae tendineae 40 as described herein. Additional rotation of the valve coil 15 may gradually capture additional leaflets 42 and/or chordae tendineae 40.

Rotation of the valve prosthesis 10, for example, rotation of the anchor 15 and/or frame structure 12, may be facilitated by the delivery device 30 described herein. For example, the inner shaft 52 and/or anchor drive shaft 180 may be rotated and rotational motion may be transmitted from the inner shaft 52 and/or anchor drive shaft 180 to the valve prosthesis 10 in order to rotate the valve prosthesis 10 around one or more of the structures on the ventricle side of the mitral valve 4 as described herein. Alternatively, or in combination, a proximal portion of the anchor 15 may be detachably coupled to an actuation arm or proximal pusher which extends through a lumen of the inner shaft 52 to a distal end thereof as described herein. The actuation arm may be rotated and rotational motion may be translated from the actuation arm to the anchor 15 in order to rotate the anchor 15 around the one or more structures on the ventricle side of the mitral valve 4 as described herein

Once the anchor 15 has been anchored adjacent to the native valve 4, the valve prosthesis 10 comprising the frame structure 12 and prosthetic valve segment 14 may be expanded at least partially within the anchor 15 as described herein. The frame structure 12 and the valve segment 14 may be deployed (e.g., expanded) simultaneously. Alternatively, or in combination, the frame structure 12 and the valve segment 14 may be deployed sequentially, for example by first expanding the frame structure 12 and then receiving the prosthetic valve segment 14 therein.

In some embodiments, the frame structure 12 and the anchor 15 may be located within the same lumen of the delivery device 30 prior to deployment. In some embodiments, the frame structure 12 and the anchor 15 may be located within different lumens of the delivery device 30.

In some embodiments, the frame structure 12 and the anchor 15 may be deployed from the same opening (e.g., a distal opening) in the delivery device 30. In some embodiments, the frame structure 12 and the anchor 15 may be deployed from different openings in the delivery device 30.

The valve prosthesis 10 may then be released from the delivery device 30. Releasing the valve prosthesis 10 from the delivery device 30 may comprise expanding the valve prosthesis 10 from the unexpanded configuration to the expanded configuration. For example, expanding the frame structure 12 and releasing the frame structure 12 may occur simultaneously as described herein. Alternatively, the frame structure 12 may be released prior to or after being expanded.

FIGS. 2A-2B show the valve prosthesis 10 fully expanded with the native valve leaflets 42 and/or chordae tendineae 40 captured between the frame structure 12 and the anchor 15. As described herein, the valve prosthesis 10 may comprise one or more valve segments 14 disposed therein to replace the native valve leaflets 42.

Although the steps above show a method of deploying a valve prosthesis 10 within a native valve 4 in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as necessary to assemble at least a part of an article.

For example, in some embodiments deploying the valve prosthesis 10 may occur in multiple steps such that a portion of the valve prosthesis 10 (e.g., anchor 15) may be deployed before another portion the valve prosthesis 10 (e.g., frame structure 12). Alternatively, or in combination, in some embodiments, deploying the anchor 15 may occur in multiple steps such that a portion of the anchor 15 may be deployed before being advanced through the native valve 4 and another portion of the anchor 15 may be deployed after being advanced through the native valve 4. Alternatively, or in combination, the delivery device 30 may be advanced from the left atrium 25 to the left ventricle 26 with the valve prosthesis 10 undeployed. In many embodiments, the frame structure 12 may be balloon-expandable and the delivery device may comprise a balloon instead of or in addition to a proximal pusher or guidewire coupling in order to expand the frame structure 12. Alternatively, or in combination, the anchor 15 may be released after the frame structure 12 has been expanded within it.

The valve prosthesis 10 may comprise a valve segment (for example, valve segment 14 shown in FIG. 2B) disposed therein. In various embodiments, valve segment is used somewhat interchangeably with prosthetic valve leaflet and generally refers to the prosthetic leaflets and frame. As used herein, “prosthetic valve” may refer to all manner of prosthetic and artificial replacement valves including tissue (biological valves), tissue-engineered valves, polymer valves (e.g., biodegradable polymer valves), and even certain mechanical valves. The valve segment can be similar to existing transcatheter valves. The valve segment can be similar to existing surgical tissue valves, and mechanical valves. At least a portion of the valve segment may be positioned within at least a portion of the valve prosthesis 10, for example with a frame structure of the valve prosthesis. The valve segment may include leaflets formed of multi-layered materials for preferential function. The valve segment may comprise at least one leaflet having an inner layer and an outer layer. The valve segment may be attached to a valve structure which is in turn connected to the valve prosthesis 10. The valve structure may be connected to the valve prosthesis 10 before or after the valve prosthesis 10 has been deployed adjacent a native valve. The valve segment may be attached directly to the valve prosthesis 10. The valve prosthesis 10 may be attached to a leaflet, for example an outer layer of a leaflet, at one or more ends of the valve prosthesis 10. The valve prosthesis 10 may be attached to a leaflet, for example an outer layer of a leaflet, at one or more intermediate portions of the valve prosthesis 10. The valve segment may comprise a plurality of leaflets. The valve segment may comprise a biocompatible one-way valve. Flow in one direction may cause the leaflet(s) to deflect open and flow in the opposite direction may cause the leaflet(s) to close.

The valve prosthesis may be substantially similar to any of the valve prostheses described in U.S. patent application Ser. No. 16/546,901 and U.S. Provisional Application Nos. 62/720,853, 62/742,043, 62/748,162, 62/755,996, 62/784,280, 62/813,963, 62/815,791, 62/820,570, 62/828,835, 62/833,425, 62/833,430, 62/851,245, 62/872,016, 61/873,454, 62/879,979, and 62/894,565, previously incorporated herein by reference for all purposes.

As can be seen in FIGS. 3A-3F, the delivery device 30 may comprise an inner shaft 52 (e.g., a delivery tube) and an optional guidewire 54 disposed within a lumen of the inner shaft 52. The inner shaft 52 can be disposed within a lumen of an outer sheath. The guidewire 54 may optionally comprise a nosecone at the distal end 211 to facilitate guidance of the guidewire 54 to the native valve 4. As shown in FIG. 3A, a guide wire 54 may be inserted through the left atrium 25 of the heart 2 via a trans septal puncture as described herein. The distal end 211 of the wire can be placed in the left ventricle 26. A proximal end of the valve prosthesis 10 may be operably coupled to the inner shaft 52 during delivery to the native valve 4 as described herein. The outer sheath and/or inner shaft 52 may be steerable.

The valve prosthesis 10 may be operably coupled to the delivery device 30 as described herein. In some embodiments, at least a portion of the valve prosthesis 10 may be directly coupled to the inner shaft 52. Alternatively, or in combination, at least a portion of the valve prosthesis 10 may be indirectly coupled to the inner shaft 52. For example, at least a portion of the valve prosthesis 10 may be coupled to a torque hub or other connector, which may be coupled to the inner shaft 52, such as the torque hub described in U.S. Patent Application No. 62/813,963, previously incorporated herein by reference for all purposes. Alternatively, or in combination, at least a portion of the valve prosthesis 10 may be directly or indirectly coupled to the guidewire 54.

Additional description for the delivery device and other similar delivery devices usable in the embodiments described herein may be found in U.S. patent application Ser. No. 16/546,901 and U.S. Provisional Application Nos. 62/720,853, 62/742,043, 62/748,162, 62/755,996, 62/784,280, 62/813,963, 62/815,791, 62/820,570, 62/828,835, 62/833,425, 62/833,430, 62/851,245, 62/872,016, 61/873,454, 62/879,979, and 62/894,565 previously incorporated herein by reference for all purposes.

The valve prosthesis 10 may, for example, comprise a frame structure 12 and an anchor 15. The anchor 15 may be directly coupled to the frame structure 12, for example at a proximal or distal end thereof. Alternatively, or in combination, the anchor 15 may be detachably coupled to the delivery device 30 prior to deployment at the native valve. The anchor 15 may comprise a deployed configuration (e.g., as shown in FIG. 3D). The frame structure 12 may have an unexpanded configuration (e.g., as shown in FIGS. 3A-3D), for example when the valve prosthesis 10 is in its unexpanded configuration, and an expanded configuration (for example, as shown in FIG. 3F), for example when the valve prosthesis 10 is in its expanded configuration. The expanded configuration may have a generally tubular expanded shape. The frame structure 12 may be configured for expanding within the native valve of the patient. In some embodiments, the unexpanded configuration may be sized and dimensioned for percutaneous insertion and the expanded configuration may be sized and dimensioned for implantation in the native valve of the patient.

The frame structure 12 may comprise a first and second opposite ends, the first end extending above a native valve and the second end extending below the native valve when the frame structure 12 is anchored to the native valve. Alternatively, the frame structure 12 may be configured to sit entirely below the native valve when the frame structure 12 is anchored to the native valve.

In some embodiments, the frame structure 12 may comprise an expanded outer periphery in the expanded configuration and a compressed outer periphery when subject to an external radial force in the unexpanded configuration. The compressed outer periphery may be smaller in diameter than the expanded outer periphery.

The valve prosthesis 10 comprising a frame structure 12 may be balloon-expandable, self-expanding, or otherwise expansible as will be understood by one of ordinary skill in the art. The frame structure 12 may, for example, comprise an expandable stent.

The delivery system 30 may comprise an inflatable balloon releasably connected to the valve prosthesis 10 and inflation of the balloon may cause expansion of the valve prosthesis 10 which comprises a frame structure 12 as described herein and in U.S. patent application Ser. No. 16/546,901 and U.S. Provisional Application Nos. 62/720,853, 62/742,043, 62/748,162, 62/755,996, 62/784,280, 62/813,963, 62/815,791, 62/820,570, 62/828,835, 62/833,425, 62/833,430, 62/851,245, 62/872,016, 61/873,454, 62/879,979, and 62/894,565 previously incorporated herein by reference for all purposes.

Alternatively, or in combination, the frame structure 12 may be self-expanding and may be maintained in the unexpanded configuration by radial constriction from the outer sheath of the delivery device. Advancement of the inner shaft 52 out of the lumen of the outer sheath may actuate the frame structure 12 into the expanded configuration as described herein and in U.S. patent application Ser. No. 16/546,901 and U.S. Provisional Application Nos. 62/720,853, 62/742,043, 62/748,162, 62/755,996, 62/784,280, 62/813,963, 62/815,791, 62/820,570, 62/828,835, 62/833,425, 62/833,430, 62/851,245, 62/872,016, 61/873,454, 62/879,979, and 62/894,565, previously incorporated herein by reference for all purposes.

The frame structure 12 and/or anchor 15 may be operably coupled to the delivery device 30 as described herein. In some embodiments, at least a portion of the frame structure 12 and/or anchor 15 may be directly coupled to the inner shaft 52. For example, a proximal portion of the frame structure 12 and/or a proximal portion of the anchor 15 may be coupled to a distal portion of the inner shaft 52. Alternatively, or in combination, at least a portion of the frame structure 12 and/or anchor 15 may be indirectly coupled to the inner shaft 52. Alternatively, or in combination, at least a portion of the frame structure 12 and/or anchor 15 may be disposed within a lumen of the inner shaft 52. In some embodiments, at least a portion of the frame structure 12 and/or anchor 15 may be directly or indirectly coupled to the guidewire 54.

The frame structure 12 may be detachably coupled to the delivery device 30 in the unexpanded configuration during delivery to the native valve. Expansion of the frame structure 12 to the expanded configuration may detach the frame structure 12 from the delivery device 30. Alternatively, or in combination, advancement of the frame structure 12 out of the delivery device 30 may detach the frame structure 12 from the delivery device 30.

In some embodiments, the frame structure 12 may be detachably coupled to and/or disposed within the delivery device 30 at a location proximal to the anchor 15. In some embodiments, the frame structure 12 may be detachably coupled to and/or disposed within the delivery device 30 at a location distal to the anchor 15. In some embodiments, at least a portion of the frame structure 12 may be detachably coupled to and/or disposed within the delivery device 30 at a location adjacent (e.g., within) the anchor 15.

In some embodiments, the anchor 15 may be detachably coupled to and/or disposed within the delivery device 30 at a location proximal to the frame structure 12. In some embodiments, the anchor 15 may be detachably coupled to and/or disposed within the delivery device 30 at a location distal to the frame structure 12. In some embodiments, at least a portion of the anchor 15 may be detachably coupled to and/or disposed within the delivery device 30 at a location adjacent (e.g., around) the frame structure 12.

As can be seen in FIGS. 3E-3F, at least a portion the frame structure 12 may be expanded within at least a portion of the deployed anchor 15 to anchor the frame structure 12 to the native valve. For example, the anchor 15 may be deployed such that it captures one or more structures therein, for example one or more chordae tendineae and/or one or more valve leaflets. Expansion of the frame structure 12, or a portion thereof, within the anchor 15 may compress the captured structures between the frame structure 12 and the anchor 15 to anchor the frame structure 12 in place.

The frame structure 12 may be configured like a stent. The frame structure 12 may, for example, comprise a scaffold in a diamond pattern formed from a shape memory material (e.g., NiTi). One of ordinary skill in the art will appreciate that many other structures, materials, and configurations may be employed for the frame structure 12. For example, the frame structure 12 may be formed of a polymer of sufficient elasticity. The frame structure 12 may be formed of a combination of metal and polymer, such as metal (e.g., shape memory material) covered in polymer. The frame structure 12 may include a variety of patterns besides diamond shapes.

The frame structure 12 may comprise a valve segment disposed therein as described herein. The valve segment may be attached to a valve structure which is in turn connected to the frame structure 12. The valve structure may be connected to the frame structure 12 before or after the frame structure 12 has been deployed adjacent a native valve. The valve segment may be attached directly to the frame structure 12. The frame structure 12 may be attached to a leaflet, for example an outer layer of a leaflet, at one or more ends of the frame structure 12. The frame structure 12 may be attached to a leaflet, for example an outer layer of a leaflet, at one or more intermediate portions of the frame structure 12.

Additional description for the frame structure and other similar frame structures usable in the embodiments described herein may be found in U.S. patent application Ser. No. 16/546,901 and U.S. Provisional Application Nos. 62/720,853, 62/742,043, 62/748,162, 62/755,996, 62/784,280, 62/813,963, 62/815,791, 62/820,570, 62/828,835, 62/833,425, 62/833,430, 62/851,245, 62/872,016, 61/873,454, 62/879,979, and 62/894,565, previously incorporated herein by reference for all purposes.

The anchor 15 may comprise a laser-cut band (e.g., as shown in FIGS. 4-9) or multi-link chain (e.g., as shown in FIGS. 10-23) having a free end. The other end of the anchor 15 may be coupled to the top (proximal end) or bottom (distal end) of the frame structure 12 as described herein. Alternatively, or in combination, the other end of the anchor 15 may not be attached to the frame structure 12 as described herein. The anchor 15 may be configured to wrap at least partially around the frame structure 12 in the deployed configuration. In some embodiments the free ends of the anchor are designed to interlock when the valve is expanded into the anchor. Locking of the free ends can increase the compressive stiffness and expansive stiffness of the anchor 15 in the deployment configuration compared to when the two ends are not joined in the deployment configuration.

The anchor 15 may be configured to be advanced from a first side of the native valve in a patient (e.g., an atrial side) to a second side of the native valve (e.g., into a ventricle of the heart) and anchor the frame structure 12 to the native valve when the frame structure 12 is in the expanded configuration adjacent the native valve in the second side of the native valve.

The anchor 15 may comprise a delivery (e.g., tightly coiled) configuration (e.g., as shown in FIG. 3A) and a deployed configuration (e.g., shown in FIG. 3D). The frame structure 12 may be configured to remain in its unexpanded configuration while the anchor 15 is in the deployed configuration. In various embodiments, the anchor 15 may be self-expanding and may move to the deployed configuration as it is removed from the delivery device. In various embodiments, the anchor 15 may be configured to self-assemble when it is deployed in the heart cavity (e.g., left ventricle). The anchor 15 may be configured to be actuated from the delivery configuration to the deployed configuration adjacent the native valve using any method or mechanism understood by one of ordinary skill in the art from the description herein. The anchor 15 may be maintained in the delivery configuration by radial constriction from an outer sheath or an inner shaft 52.

In some embodiments, the anchor 15 may be configured to wrap at least partially around a distal portion of the delivery device 30, for example around the inner shaft 52 and/or a delivery gear (such as delivery gear 180) as described herein. In some embodiments, a distal portion of the inner shaft 52 may comprise the delivery gear. In some embodiments, the delivery gear may comprise a lumen through which the guidewire may be threaded.

In some embodiments, the anchor 15 may be actuated from the delivery configuration to the deployed configuration on a first side of the native valve prior to being advanced to a second side of the native valve. For example, the anchor 15 may be deployed in a left atrium of a heart prior to being advanced to a left ventricle of the heart as described herein.

Alternatively, the anchor 15 may be actuated from the delivery configuration to the deployed configuration on a second side of the native valve after being advanced to the second side from a first side of the native valve. For example, anchor 15 may be advanced from a left atrium of a heart prior to being deployed in a left ventricle of the heart.

The anchor 15 may be detachably coupled to a proximal or distal portion of the frame structure 12 as described herein. Alternatively, or in combination, the anchor 15 may be detachably coupled to the delivery device 30 in the delivery configuration during delivery to the native valve. For example, the proximal end of the anchor 15 may be detachably coupled to the inner shaft 52 of the delivery device 30 by radial constriction from the outer sheath. Retraction of the outer sheath away from the proximal end of the anchor 15 (or, similarly, extrusion of the distal end of the anchor 15 out of an opening in the outer shaft) may detach the anchor 15 from the delivery device 30. Alternatively, or in combination, the proximal end of the anchor 15 may be detachably coupled to the inner shaft 52 of the delivery device 30 by an attachment element. Alternatively, or in combination, the proximal end of the anchor 15 may be detachably coupled to the inner shaft 52 of the delivery device 30 by a weak adhesive.

In some embodiments, the anchor 15 may be disposed in a lumen of the inner shaft 52. The anchor 15 may or may not be coupled to the inner shaft 52. The anchor 15 may be maintained in the delivery configuration by radial constriction from the inner shaft 52. Advancement of the anchor 15 out of the inner shaft 52, for example out of a distal opening or a lateral side opening (e.g., side port 214 shown in FIG. 3B) of the inner shaft 52, may actuate the anchor 15 into the deployed configuration. The proximal end of the anchor 15 may be detachably coupled to an actuation arm (e.g., proximal pusher) which may be disposed within the lumen of the inner shaft 52 and extend towards a proximal end of the delivery device 30.

In various embodiments, the anchor 15 may comprise a curved shape in the deployed configuration. In various embodiments, the anchor 15 may be formed as a flat curve (in the deployed configuration) whereby the loops generally are positioned within the same plane (the plane being perpendicular to a longitudinal axis). In various embodiments, the anchor 15 may be formed as a three-dimensional curve (in the deployed configuration) whereby the loops generally are positioned out of plane with one another.

The anchor 15 may comprise a spiral shape in the deployed configuration. As used herein, a spiral or spiral shape may comprise a curve which emanates from a point (e.g., a central point) having a continuously increasing or decreasing distance from the point. The spiral or spiral shape may be two-dimensional (e.g., planar) or three-dimensional. In some embodiments, the anchor 15 may comprise one or more spiral portions as described herein.

In various embodiments, the anchor may have a spiral-shaped deployed configuration. In various embodiments, spiral refers to a shape with windings about a central axis. The spiral may be continuous. The windings may gradually widen (or tighten) along the length. The spiral may be formed in a flat plane perpendicular to the central axis. In various embodiments, the anchor may have a deployed configuration that is not formed in a flat plane, or in other words the deployed shape is formed in a three-dimensional and/or non-degenerate space. In various embodiments, the anchor may have a conical-shaped deployed configuration including, but not limited to, tubular, conical, frustoconical, and/or helical shapes.

The anchor 15 may comprise a free end 22. The free end 22 of the anchor 15 may be sized and dimensioned for insertion through the native valve, for example through tissue at or near a commissure of the native valve or through the valve opening itself. In some embodiments, the free end 22 may comprise an atraumatic tip to avoid or reduce the risk of injury to the native valve tissue and leaflets. For example, the free end may comprise a blunt end, a ball tip, a curved tip (e.g. J-tip or pigtail), or other atraumatic shapes. Alternatively, the free end 22 may be configured for piercing tissue. In various embodiments, the free end 22 may be shaped and configured to reduce the risk of counterrotation. For example, the tip 22 may have a curled end to cause the free end 22 to snag tissue (e.g., chordae) if it is rotated in a direction opposite the anchoring rotation.

The free end 22 of the anchor 15 may extend radially outward from the frame structure 12, and in particular from the remainder of the anchor 15. The other end of the anchor 15 may be coupled to the top or bottom of the frame structure 12 as described herein. Alternatively, or in combination, the other end of the anchor 15 may not be attached to the frame structure 12 as described herein. The free end 22 of the anchor 15 may facilitate capturing of the valve leaflets and/or chordal tendineae within the sweep of the free end during rotation as described herein. During rotation of the anchor 15, the leaflets and/or chordae tendineae may be captured by the free end 22 and trapped between the valve frame structure 12 and an interior surface of the anchor 15.

The anchor 15 may comprise one or more loops. For example, the anchor 15 may comprise a plurality of loops in the deployed configuration, which may increase the radial strength of the anchor by increasing friction and addition structural support. The one or more loops of the anchor 15 may spiral radially outward from a central point or central axis of a spiral shape, for example along an axis which is coaxial with a longitudinal axis of a delivery device 30 such that the anchor lies approximately along a plane perpendicular to the longitudinal axis of a delivery device. In some embodiments, the one or more loops of the anchor 15 may comprise one or more spaces therebetween. The spaces may facilitate movement of the captured tissue (e.g. chordae and/or leaflets) from the free end 22 to the center of the spiral structure during rotation of the anchor 15 as described herein.

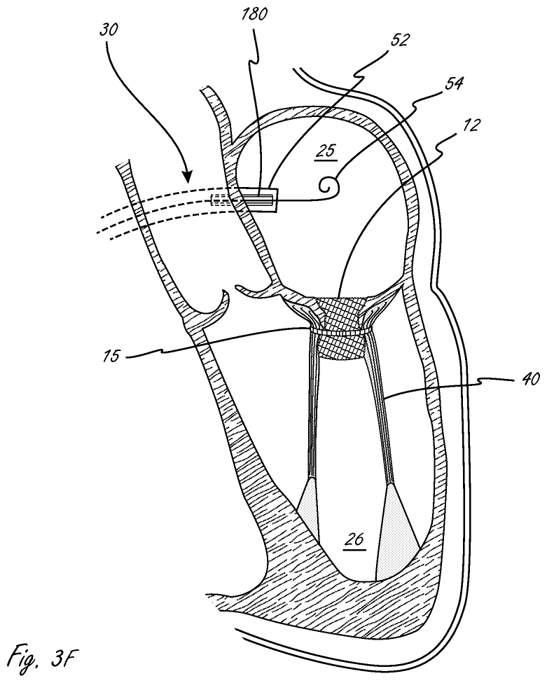

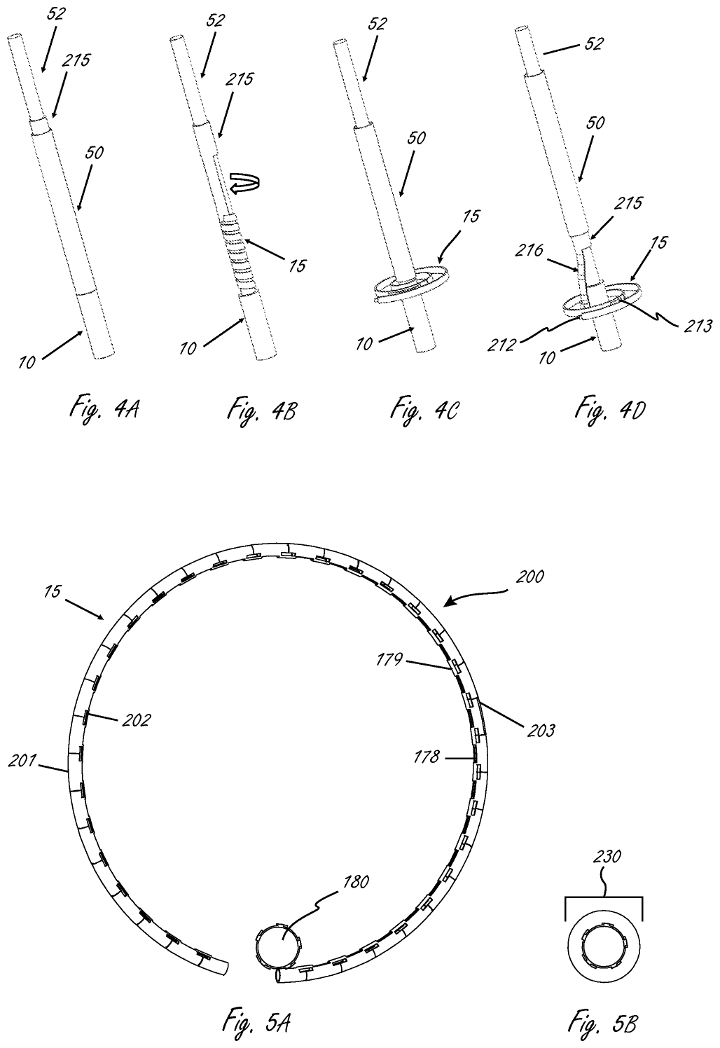

FIGS. 4A-4D shows another embodiment of the deployment of the anchor 15 by a “flat screw deployment” delivery device 30. The anchor 15 may be substantially similar to any of the anchors described herein, for example any of the anchors 15 shown in FIGS. 5A-24. In some embodiments, the anchor 15 may be coupled to the delivery device 30 and/or a frame structure 12 as described herein. The frame structure 12 may be substantially similar to any of the frame structures described herein. The delivery device 30 may comprise an inner shaft 52 and an outer shaft 50. The inner shaft 52 may be substantially similar to any of the inner shafts described herein. The outer shaft 50 may be substantially similar to any of the outer shafts described herein. The anchor 15 may be coupled to the inner shaft 52 as described herein. The frame structure 12 may be coupled to the inner shaft 52, for example around the inner shaft 52 or at a distal end of the inner shaft 52, as described herein.

The delivery device 30 may further comprise an anchor drive shaft 215. The anchor 15 may be disposed on or around the anchor drive shaft 215 in a screw-like undeployed configuration. FIG. 4B shows an exemplary anchor 15 disposed around anchor drive shaft 215 in a screw-like undeployed configuration with the outer shaft 50 removed in order to show the internal components of the delivery device 30 in relation to the undeployed valve prosthesis 10. At least a portion of the anchor drive shaft 215, for example a distal end (e.g., deployment drive 216), may be operably coupled to the anchor 15. The anchor drive shaft 215 may be rotatable relative to the outer shaft 15. The anchor drive shaft 215 may be configured to transmit rotational motion and/or torque to the anchor 15 in order to rotate the anchor out of the delivery device 30 and/or around the one or more structures of the native valve as described herein.

Deployment of the anchor 15 from the delivery device 30 may be facilitated by combined retraction of at least a portion of the outer shaft 50 relative to the inner shaft 52 to form or expose a lateral opening in the delivery device 30 and rotation of an anchor drive shaft 215 relative to the outer shaft 50 and/or inner shaft 52.

The anchor 15 may be actuated from a delivery configuration (shown in FIG. 4B) to a deployed configuration (shown in FIG. 4C). The delivery configuration may be substantially similar to any of the delivery configurations described herein. For example, the anchor 15 may comprise a compact screw-like spiral shape when disposed around the inner shaft 52 in the delivery configuration. The deployed configuration may be substantially similar to any of the deployed configurations described herein. For example, the anchor 15 may comprise a flat spiral shape in the deployed configuration and at least a portion of the spiral anchor 15 may be disposed about or proximal to a distal end of the inner shaft in the deployed configuration. In some instances, the anchor 15 may comprise a plurality of intermediate deployed configurations (e.g., as shown in FIG. 3C). For example, the anchor 15 may be progressively deployed from the initial delivery configuration to a final deployed configuration though one or more intermediate deployed configurations in which at least a portion (e.g., a distal portion) of the anchor 15 has a flat spiral shape while another portion (e.g., a proximal portion) remains wound around the inner shaft 52 in a compact screw-like spiral shape. As the anchor 15 continues to deploy towards the final deployed configuration, more and more of the anchor 15 unwinds from the compact screw-like spiral shape into the flat spiral shape until, finally, the entire anchor 15 is deployed.

The outer shaft 50 may be retracted such that a lateral opening is formed and the distal end 22 of the anchor 15 is exposed. Continued rotation of the anchor drive shaft 215 may actuate the anchor 15 out of the opening S into the deployed configuration through its coupling with deployment drive 216.

In some embodiments, the outer shaft 50 may be moved back and forth over the anchor drive shaft 215 prior to, during, or after rotation of the anchor drive shaft 215 in order to “ratchet” incremental portions of the anchor 15 out of the delivery device 30 and away from the inner shaft 52. For example, the anchor may be deployed by a retraction of the outer shaft 52 followed by a series of rotations of the anchor drive shaft 215 followed by an advancement of the outer shaft 215, which may be repeated as needed to deploy the fully anchor 15.

The anchor 15 may be releasably coupled to the deployment drive 216.

Once the anchor 15 is in the deployed configuration, the deployment drive 216 may remain connected until the anchor 15 is fully secured around the diseased valve and fully deployed implant valve. The deployment drive 216 may be used to translate the anchor 15 distally such that it sits at least partially around the frame structure 12 (as shown in FIG. 4D). The anchor 15 may then be advanced through the native valve and rotated around one or more native structures as described herein.

Alternatively, the deployment drive 216 may be disconnected from the anchor 15 prior to the anchor 15 being secured to the one or more structures of the diseased valve.

The anchor 15 may be deployed from the delivery device 30 in the left atrium of the heart and advanced into the left ventricle through the diseased mitral valve as described herein. Alternatively, the anchor 15 may be deployed from the delivery device 30 in the left ventricle of the heart as described herein. Alternatively, the anchor 15 may be partially deployed in the left atrium, advanced into the left ventricle, and then fully deployed in the left ventricle as described herein.

In some embodiments, deployment of the anchor 15 and capture of the one or more structure of the native valve may occur in a stepwise fashion. For example, the anchor 15 may be deployed before being rotated to capture the one or more structures.

In some embodiments, deployment of the anchor 15 and capture of the one or more structure of the native valve may occur simultaneously. For example, rotation of the anchor drive shaft Y may rotate the anchor 15 out of the delivery device 30. If deployed in the left ventricle, the free end 22 of anchor 15 may be rotated around the one or more structures as the anchor 15 is rotated out of the delivery device.

The distal end of the anchor 15 may comprise a key 212 configured to slide into a complementary lock 213 located on the band of the anchor 15. When the anchor 15 is fully deployed and wrapped around the frame structure 12 and the diseased valve, the key 212 may slide over the band of the anchor 15 until it falls into place within the lock 213. Once engaged, the key 212 and lock 213 may hold the anchor 15 in place against the one or more structures of the native valve. It will be understood by one of ordinary skill in the art from the teachings herein that the respective locations of key 212 and lock 213 may be configured to lock the anchor 15 into the fully deployed configuration, the fully undeployed configuration, or any intermediate configuration therebetween. It will be understood by one of ordinary skill in the art from the teachings herein that any number of key and lock elements may be placed on the anchor 15 in order to allow for one or more locked configurations as desired. It will be understood by one of ordinary skill in the art from the teachings herein that the key and lock may be replaced or added to any locking mechanisms understood to one of skill from the teachings herein. For example, a frictional band may replace or be added to the key and lock locking mechanism.

FIGS. 5A-24 show various anchor embodiments which may be deployed using the delivery devices shown in FIGS. 3A-3F or FIGS. 4A-4D or with any of the delivery devices described herein or which will be known to one of ordinary skill in the art based on the teachings herein. The anchor 15, as disclosed herein, can have a helical delivery configuration when wrapped around a delivery gear and a circular delivered configuration when released from the delivery gear. The anchor can have a low compressive stiffness and low expansive stiffness when in a delivery configuration relative to a high expansion stiffness when expanded past the delivery radius of the delivery configuration.

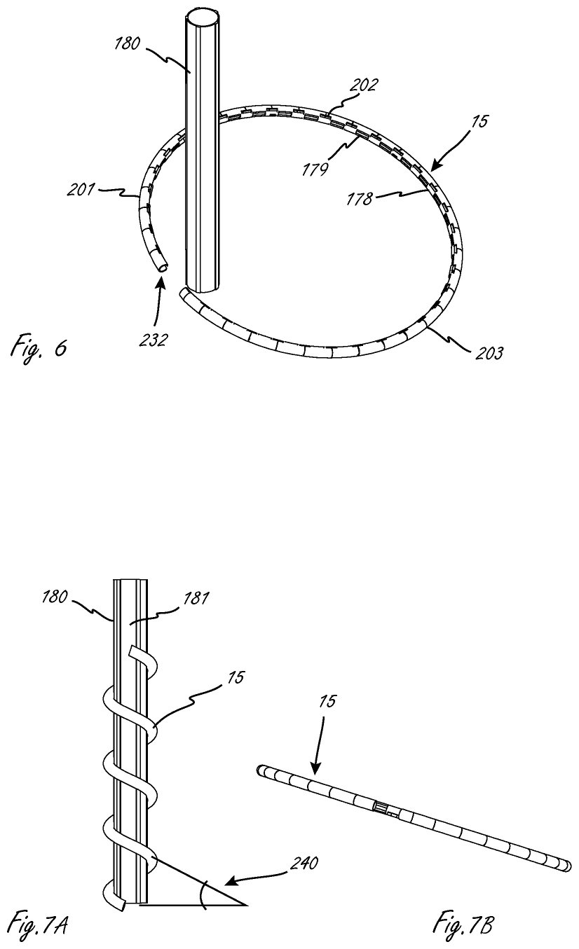

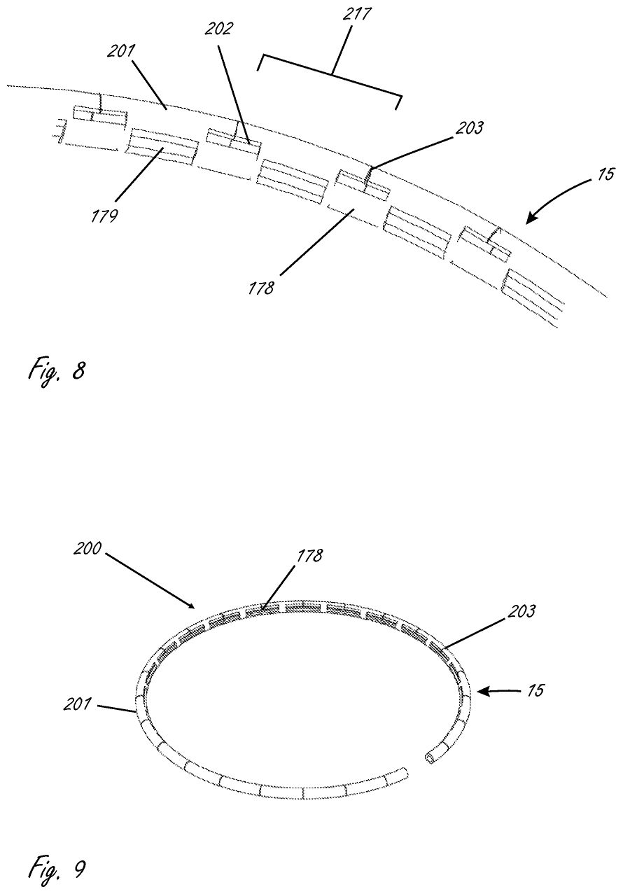

FIG. 5A shows a top view of a laser-cut anchor 15 in a deployed configuration. FIG. 5B shows the circumference of the delivery configuration around an anchor delivery gear 180. FIG. 6 shows a perspective view of the laser-cut anchor 15 of FIG. 5A after deployment from a delivery configuration. FIG. 7A shows a perspective view of the laser-cut anchor 15 of FIG. 5A in a delivery configuration around a delivery gear 180, the delivery gear 180 comprising a delivery tooth 181 configured to hold the anchor 15 in the delivery configuration. FIG. 7B shows a side view of the laser-cut anchor 15 of FIG. 5A in the deployed configuration. FIG. 8 shows a close perspective view of the laser-cut anchor 15 of FIG. 5A in the deployed configuration, highlighting the gear interface 179 of the laser cut anchor 15. The anchor 15 may comprise a tubular anchor body 232 having one or more cuts therein to provide for flexibility, tensioning and/or shaping, and/or engagement with a delivery device. The delivery gear 180 may lack a delivery tooth, wherein the interface with the anchor moves the anchor off of the delivery gear 180 by friction. The helical configuration of the anchor 15 around the delivery gear 180 may comprise delivery pitch angles 240 between 5 degrees and 85 degrees, as illustrated in FIG. 7A. The delivery pitch angle 240 can be within a range bounded by any two of the following values: 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, and 85 degrees.

As shown in FIGS. 5A, 5B, 6, 7A, 7B, and 8, the anchor 15 can comprise a series of pseudo units 217 partially separated from each other by outer radius limiter cuts 203 in a single band of the anchor 15 so that the pseudo units 217 are connected by a tie band 178. That is, the pseudo units 217 are connected by the material at an inner radius of the anchor 15 corresponding to the tie band 178. The outer radius limiter cuts 203 are axial cuts that are formed partially through the material of the single band to allow the anchor 15 to flex and transition between the delivery configuration and the deployed configuration. Each pseudo unit 217 can comprise a gear interface 179 on its inner edge. As can be seen in FIG. 8, between each pseudo unit 217 can be a tie relief cut 202 and/or an outer radius limiter 203 on the outer edge of the anchor 15 to allow the anchor 15 to flex into a tight spiral of a delivery configuration. The spine 201 of the anchor 15 may restrict the anchor 15 from flexing beyond the curved shape of the deployed configuration. As can be seen in FIGS. 5A and 6, the gear interface 179 of each pseudo unit 217 can attach to a tooth 181 of a delivery gear 180 in a delivery configuration. In some embodiments, as can be seen in FIGS. 6 and 8, the series of pseudo units 217 cut into a single band can comprise a gear interface 179 on the inner edge of the anchor 15, cut so as to flank the tie band 178.

The anchor 15 may comprise a series of pseudo-units 217 extending between outer radius limiter cuts 203 made in the spine 201 of a tubular anchor body. The outer radius limiter cuts 203 may be configured to provide flexibility/compliance (i.e., degrees of freedom) and allow the anchor 15 to wrap into a screw-like delivery configuration, with a small outer radius, or open into a flat spiral or circular-shaped deployed configuration, with a larger outer radius. The delivery configuration may have a minimum outer radius and the deployed configuration may have a maximum outer radius. The outer radius limiter cuts 203 may be configured such that, when the edges of cuts defining the pseudo-units 217 contact one another upon being deployed into the deployed configuration, the outer radius of the anchor 15 is prevented by the spine 201 from expanding beyond the maximum outer radius of the deployed configuration.

The anchor 15 may comprise a plurality of intermediate deployed configurations. For example, the anchor 15 may be progressively deployed from the initial delivery configuration (e.g., as shown in FIG. 7A) to a final deployed configuration (e.g., as shown in FIG. 7B) though one or more intermediate deployed configurations in which at least a portion (e.g., a distal portion) of the anchor 15 has a flat spiral or circular shape while another portion (e.g., a proximal portion) remains wound around the delivery gear 180 in a compact screw-like spiral shape (e.g., as shown in FIG. 3C). As the anchor 15 continues to deploy towards the final deployed configuration, more and more of the anchor 15 unwinds from the compact screw-like spiral shape into the flat spiral or circular shape until, finally, the entire anchor 15 is deployed.

The anchor 15 may comprise one or more tie band relief cuts 202 defining a tie band 178 along an inner surface of the anchor 15. The tie band 178 may extend along an inner surface of the anchor 15 and provide tension to the inner circumference to the anchor 15. The tie band 178 may be configured to tension the inner circumference of the anchor 15 in order to urge the anchor 15 to move from the delivery configuration to the deployed configuration with little or no force applied to the anchor 15. For example, when the anchor 15 is released from radial constriction (e.g., from an outer shaft or inner shaft of a delivery device as described herein), the tie band 178 may be biased to unwrap from the screw-like delivery configuration into the flat spiral deployed configuration. Once the anchor 15 is in the expanded deployed configuration, the tie band 178 can apply a radial expansion force to maintain the anchor 15 in the expanded deployed configuration. The outer radius limiter cuts 203 may prevent the tie band 178 from expanding the anchor 15 beyond the desired deployed configuration as described herein. The tie bands act as tension member in opposition to the compressive forces born by the outer radius limiter cut surfaces when the anchor is expanded past its delivered configuration.

In some embodiments, the tie band 178 may run parallel to the inner circumference of the anchor 15 comprising a pitch angle of the tie band between tie band stops 239 of 0 as illustrated in FIG. 10. In some embodiments, the tie band 178 may wrap around the anchor 15, forming a helical member between the tie band stops 239, for example, in order to reduce the force require to wrap the anchor 15 into a screw-like helical formation in the delivery configuration and/or increase the distance between pseudo-units when the anchor is in the helical delivery configuration. The pitch angle of the tie band between tie band stops 239 can be within a range bounded by any two of the following values: 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, and 180 degrees.

The tension provided by the tie band 178 may be related to the stiffness of the anchor body material and/or the thickness of the tie band 178, as will be understood by one of ordinary skill in the art based on the teachings herein. In some embodiments, the anchor body material may comprise biocompatible polymers, biocompatible metals, nitinol, PET, polyamide, PEEK, Ultem, polypropylene, stainless steel, titanium, etc.

The anchor 15 may be configured such that when it is compressed by radial constriction (e.g., by an outer shaft of a delivery device as described herein to retain its delivery configuration) it is relatively compliant and when it is compressed e.g., in a delivered configuration to a delivery configuration and it is relatively rigid when expanded past a delivered configuration. The rigidity of the anchor 15 in the deployed configuration may enable the anchor 15 to retain a frame structure as described herein.

The anchor 15 can be deployed prior to the frame structure 12 of the valve prosthesis 10. The frame structure 12 may be in a delivery configuration within an inner sheath 52 when the anchor 15 is deployed. The anchor 15 can be deployed within the left ventricle 26, adjacent one or more chordae tendineae 40 and/or one or more native leaflets 42. The anchor may be rotated to capture and anchor the native chordae 40 and/or native leaflets 42. The anchor 15 may be deployed distal to the frame structure 12 and secured to the native chordae 40 and/or native leaflets 42 when the frame structure 12 is deployed.

The anchor 15 may comprise one or more cuts defining a gear interface 179 on an inner surface of the anchor body which correspond to a delivery tooth 181 of a delivery gear 180 of a delivery device. The delivery device may be substantially similar to any of the delivery devices described herein or known to one of ordinary skill in the art. For example, the delivery device may comprise an inner shaft disposed within a lumen of an outer shaft as described herein. The delivery gear 180 may be coupled to a distal end of the inner shaft such that movement of the inner shaft relative to the outer shaft correspondingly moves the delivery gear 180 relative to the outer shaft. In some embodiments, the distal end of the inner shaft may comprise a delivery gear 180. Translation and/or rotation of the delivery gear 180 (which may be attached to or comprise a distal end of the inner shaft) may deploy the anchor 15 as described herein.

In some embodiments, each pseudo-unit 217 of the anchor 15 may comprise one or more cuts defining a gear interface 179 on an inner surface of the anchor body. In some embodiments, at least one pseudo-unit 217 of the anchor 15, but not all pseudo-units 217, may comprise one or more gear interface 179 cuts. In some embodiments, no gear interface 179 cuts may be required for the anchor 15 to interface with the delivery device. It will be understood by one of ordinary skill in the art based on the description herein that the number and placement of the gear interface cuts 179 (if any) may vary as desired or needed, for example depending on the delivery device utilized to deploy the anchor 15. In one embodiment of such a device the delivery drive interfaces with anchor 15 by friction.

The anchor 15 may comprise one or more loops as described herein. In some embodiments, the anchor 15 may comprise a single loop. In some embodiments, the anchor 15 may comprise two loops. In some embodiments, the anchor 15 may comprise more than 2 loops. In some embodiments, the anchor 15 may comprise less than one loop (e.g., the anchor 15 may comprise a 270 degree or 315 degree spiral). In some embodiments, the anchor 15 may comprise more than 1 and less than 2 loops (e.g., the anchor 15 may comprise 360 degrees, 385 degrees, 405 degrees, 440 degrees, 450 degrees, 495 degrees, 540 degrees, 585 degrees, 630 degrees, or 675 degrees).

In some embodiments, the anchor 15 may be configured to be locked in the deployed configuration. For example, one or more locking mechanisms may be disposed on the anchor 15 to lock the anchor 15 into place as described herein. In some instances, the locking mechanisms may be disposed on the two ends of the spiral anchor 15 and configured to engage one another (e.g., as a lock and key, etc.) to lock the anchor 15 in the deployed configuration. Locking of the two ends can increase the compressive stiffness and expansive stiffness of the anchor 15 in the deployment configuration compared to when the two ends are not locked in the deployment configuration. It will be understood by one of ordinary skill in the art that any locking mechanism(s) described herein or known to one of ordinary skill in the art based on teachings herein may be used as desired.

The anchor 15 may be manufactured in a variety of ways as will be understood by one of ordinary skill in the art based on the description herein. For example, a straight tubular anchor body may be cut to a desired length. The tie band relief cuts 203 and/or gear interface slots 179 may be cut into the body of the anchor 15, for example, using a laser. The tubular anchor body may be then be shape-set (e.g., using a heat set material and applying heat) into the desire anchor shape (e.g., a flat spiral). The outer radius limiter cuts 203 may then be made (e.g., with a laser) on an outer surface of the spiral-shaped anchor body so as to define the pseudo-units 217 and enable collapse while preventing further expansion of the anchor 15. In some embodiments, a wire (e.g., a nitinol wire), may be threaded through a lumen of the tubular anchor body in order to lock the anchor 15 when in the deployed configuration.

FIG. 9 shows a view of a laser-cut anchor 15 in a deployed configuration. FIG. 10 shows a close view of the laser-cut anchor 15 of FIG. 9, the anchor 15 comprising a tie band 178 flanked by cuts that can serve as tie relief cuts 202 and gear interfaces 179. The anchor 15 may be substantially similar to the anchor shown in FIGS. 5A-8 except that the tie band 178 may be thinner and the anchor 15 may have cuts that serve as both tie relief cuts 202 and gear interfaces 179, instead of having distinct gear interface cuts as shown in FIGS. 5A-8. The anchor 15 may comprise a plurality of outer radius limiter cuts 203 defining a plurality of pseudo-units 217 as described herein. The thinner band can require less force, in the form of radial constriction by the inner sheath 52, to maintain the anchor 15 in a delivery configuration than a thicker band. The thinner band may be more flexible when coupled to an anchor delivery gear 180 in a delivery configuration, which may aid in maneuvering of the delivery device.

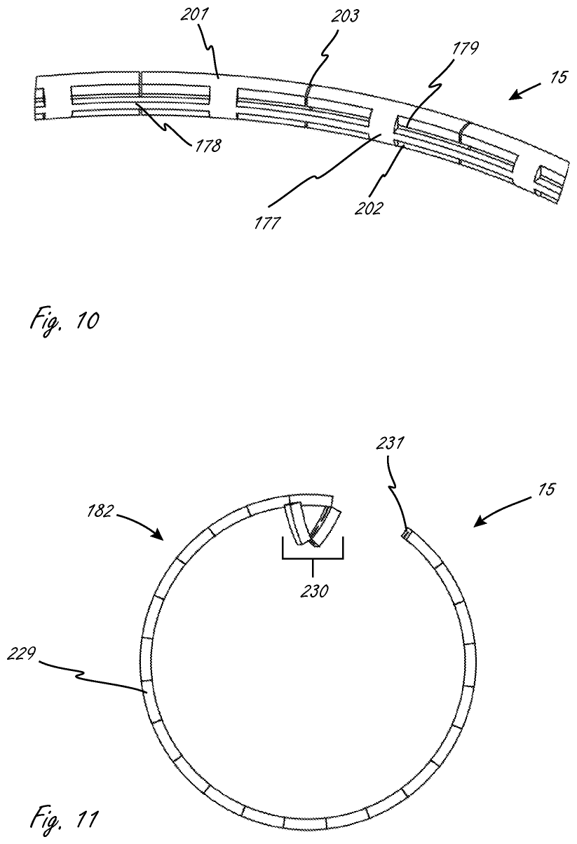

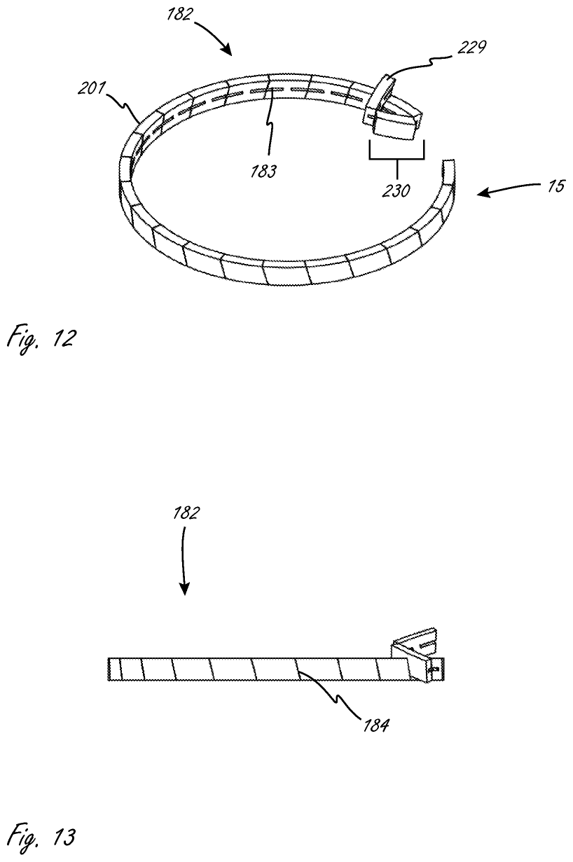

FIG. 11 shows a top view of an anchor 15, specifically a multi-link anchor 182, in a (nearly) deployed configuration. FIG. 12 shows a perspective view of the multi-link anchor 182 of FIG. 10 highlighting the tie band wire slots 183 of the anchor 15. FIG. 13 shows a close side view of the multi-link anchor 182 of FIG. 11 highlighting the angled edges 184 of each unit 229 (also referred to herein as links) configured to facilitate wrapping into a delivery configuration. FIG. 14 shows a close perspective view of the multi-link anchor 182 of FIG. 11 showing the tie band wire slot 183 of the anchor 15. The anchor 15 may comprise a series of units 229 connected by an internal wire. Each unit 229 is a single entity (thereby distinguishing them from pseudo units) but can be coupled to each other using a tie band (e.g., wire) or other coupling structure(s). Each unit 229 may be substantially rectangular, square, quadrangular, spherical, oval, or the like. The pseudo units 217 and units 229 (also referred to has links) described herein can each be referred to generally as segments.

As shown in FIGS. 11, 12, 13 and 14, the anchor 15 can comprise a series of units 229 (also referred to herein as links) connected by a tie band. In some embodiments, the tie band comprises a wire, which is held within a lumen 231 of each unit 229. The side edges of each unit 229 adjacent another unit 229 can comprise an edge/fold angle 184. The edge or fold angle 184 may be slanted such that the anchor 15 may be delivered as a spiral with a minimum diameter within a delivery device in its delivery configuration. Each unit 229 can comprise a tie band wire slot 183 to allow the wire, within a lumen of each unit 229, to flex through the tie band wire slot 183 when the anchor 15 is in the tight spiral of the delivery configuration. The wire may comprise a pre-formed shape configured to prevent flex beyond of the anchor 15 the curved shape of the deployed configuration. For example, the wire may comprise a shape-memory material configured to be shape-set into a curved shape corresponding to the deployed configuration of the anchor 15 such that the wire is biased to return to the curved shape when the anchor is deployed from the delivery configuration to the deployed configuration.

The anchor 15 may comprise a series of units 229 connected in series by a wire threaded through a lumen of each unit 229. The units 229 may abut one another in such a way that when the anchor 15 is in the deployed configuration, the wire length and contact of angled edges 184 between units 229 are configured to provide flexibility/compliance (i.e., degrees of freedom) and allow the anchor 15 to wrap into a screw-like helical delivery configuration, with a small outer radius, or open into a flat spiral or circular-shaped deployed configuration with a larger radius. The delivery configuration may have a minimum outer radius and the deployed configuration may have a maximum outer radius. The wire may be configured to bulge radially inwards through the tie band wire slots 183 when the anchor 15 is in the delivery configuration to relieve tension on the wire and enable wrapping of the anchor 15 into the delivery configuration. The angled edges between units and wire length may be configured such that, when the edges of the units contact one another upon being deployed into the deployed configuration, the outer radius of the anchor 15 is prevented by the wire and the alignment of the units from expanding beyond the maximum outer radius of the deployed configuration.

The anchor 15 may comprise a plurality of intermediate configurations during deployment (e.g., as shown in FIGS. 11 and 12). For example, the anchor 15 may be progressively deployed from the initial delivery configuration to a final deployed configuration though one or more intermediate deployed configurations in which at least a portion (e.g., a distal portion) of the anchor 15 has a flat spiral shape while another portion (e.g., a proximal portion) remains wound in a compact screw-like spiral shape. In some embodiments, the anchor 15 is held in the compact screw-like spiral shape by an external radial constriction provided by a lumen of a delivery device. As the anchor 15 is released from a lumen of a delivery device, it is released into a deployed configuration as more and more of the anchor 15 unwinds from the compact screw-like spiral shape into the flat spiral shape until, finally, the entire anchor 15 is deployed.

The anchor 15 may be configured such that when it is compressed by radial constriction (e.g., by an outer shaft or an inner shaft of a delivery device as described herein to retain its delivery configuration) it remains relatively compliant and when it is uncompressed (e.g., in the deployed configuration) it is relatively rigid relative to increasing in diameter. The rigidity of the anchor 15 in the deployed configuration may enable the anchor 15 to retain a frame structure as described herein. In some embodiments, the rigidity of the anchor 15 in the deployed configuration may, at least in part, be provided by tension in the tie band wire on the inner radius provided therein and compression between members at the outer radius limiter 203. The anchor may have a low compressive stiffness and low expansive stiffness when in a delivery configuration, and a high expansion stiffness when expanded past a delivery radius. The anchor may have a helical delivery configuration and a circular deployed configuration.

In some embodiments, the tic band wire may run parallel to the inner circumference of the anchor 15. In some embodiments, the tie band wire may run at an angle to the inner circumference of the anchor 15, for example, in order to help the anchor 15 assume a screw-like helical formation in the delivery configuration.

The tension provided by the tie band wire may be related to the stiffness of the anchor body material and/or the thickness of the tie band wire, as will be understood by one of ordinary skill in the art based on the teachings herein

The anchor 15 may comprise one or more tie band wire slots 183 in each unit 229 along an inner surface of the anchor 15. The tie band wire slots 183 may extend along an inner surface of the anchor 15 and provide a gap exposing a lumen 231 of each unit 229 to allow the tie band wire to bulge through the tie band wire slot when the anchor 15 is in a spiral delivery configuration with a minimum outer radius. Bulging of the tie band wire through the tie band wire slots may facilitate wrapping of the anchor 15 into the delivery configuration as described herein.

The tension provided by the tie band wire may be related to the stiffness of the anchor body material and/or the thickness of the tie band, as will be understood by one or ordinary skill in the art based on the teachings herein. In some embodiments the tie band wire material may comprise biocompatible metals or biocompatible polymers. In some embodiments, the biocompatible polymers can comprise polyethylenterephthalate (PET), polytetrafluoreoethylene (PTFE), polyethylene (PE), etc. In some embodiments, the biocompatible metals can comprise nitinol, titanium, stainless steel, etc.

The anchor 15 may comprise one or more loops as described herein. In some embodiments, the anchor 15 may comprise a single loop. In some embodiments, the anchor 15 may comprise two loops. In some embodiments, the anchor 15 may comprise more than 2 loops. In some embodiments, the anchor 15 may comprise less than one loop (e.g., the anchor 15 may comprise a 270 degree or 315 degree spiral). In some embodiments, the anchor 15 may comprise more than 1 and less than 2 loops (e.g., the anchor 15 may comprise 360 degrees, 385 degrees, 405 degrees, 440 degrees, 405 degrees, 450 degrees, 495 degrees, 540 degrees, 585 degrees, 630 degrees, or 675 degrees).

In some embodiments, the anchor 15 may be configured to be locked in the deployed configuration. For example, one or more locking mechanisms may be disposed on the anchor 15 to lock the anchor 15 into place as described herein. In some instances, the locking mechanisms may be disposed on the two ends of the anchor 15 and configured to engage one another (e.g., as a lock and key, etc.) to lock the anchor 15 in the deployed configuration. It will be understood by one of ordinary skill in the art that any locking mechanism(s) described herein or known to one of ordinary skill in the art based on teachings herein may be used as desired.

Note that in the example of FIGS. 11-14, in some variations, the units 229 may be held together by a live hinge where the units 229 are linked together by a thin flexible hinge made of the same material as the units 229. In such variations, the units 229 correspond to pseudo units. In such cases, a tie band wire may not be necessary to hold the units 229 together. However, a tie band spring wire may be used to provide a radial expansion force to maintain the anchor in the deployed configuration.

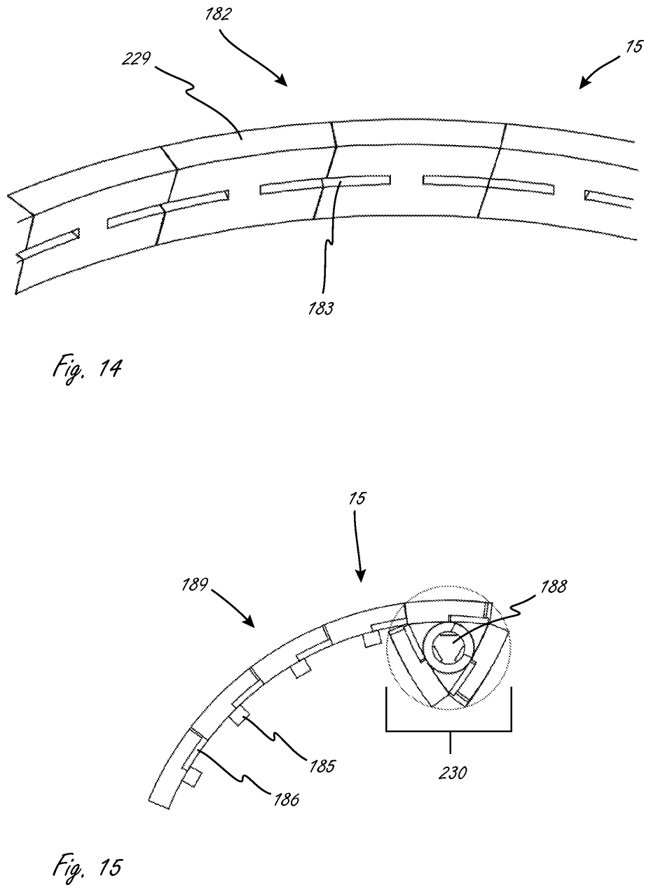

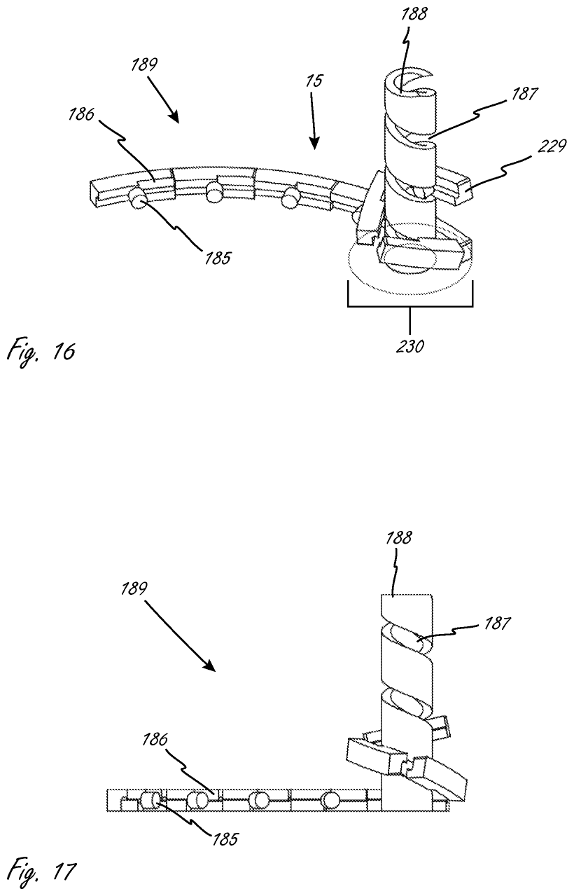

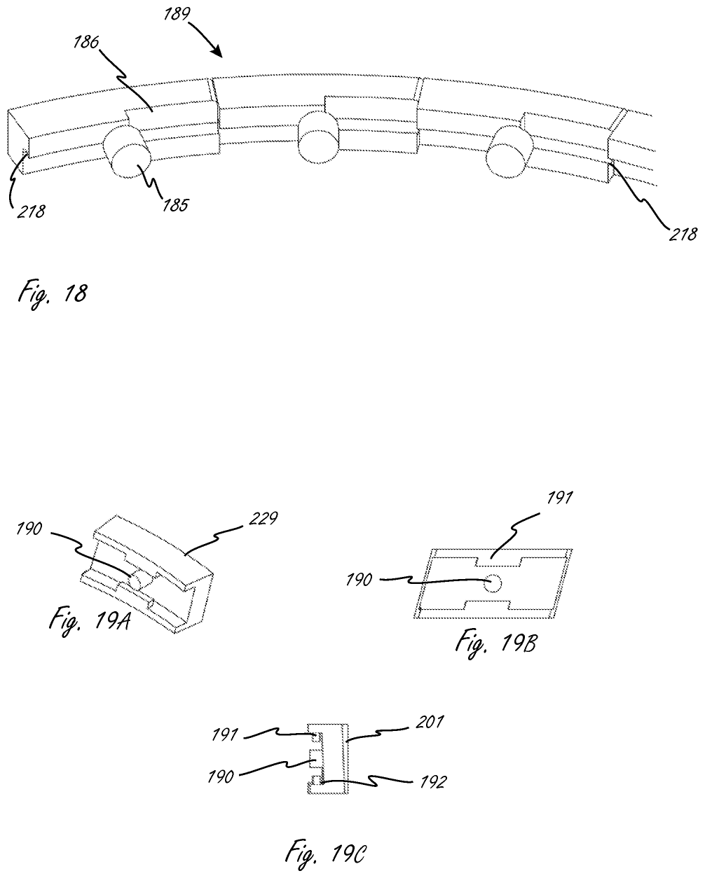

FIG. 15 shows a top view of a multi-link anchor 189 comprising unit drive pins 185 on each unit 229 of the anchor 15, the unit drive pins 185 being configured to uncouple to a delivery drive 188 as the anchor 15 is deployed from a delivery configuration around the delivery drive 188 to a deployed configuration. FIG. 16 shows a perspective view of the anchor 15 of FIG. 15 highlighting the placement of the drive pins 185 in a delivery pin guide 187 of the delivery drive 188. FIG. 17 shows a side view of the anchor 15 of FIG. 15 with pins 185 showing the deployment of the anchor 15 from the delivery configuration around the delivery drive to the deployed configuration. FIG. 18 shows a close perspective view of the anchor 15 of FIG. 15, the anchor 15 comprising tie band wire reliefs 186 present on each unit of the anchor.

As shown in FIGS. 15, 16, 17, and 18 the anchor 15 can comprise a series of units 229 connected by a tie band. In some embodiments, the tie band may comprise a wire. Each unit can comprise a tie band wire relief 186 configured to hold the tie band wire and a unit drive pin 185 to fit within a groove 187 of a delivery pin guide 188 when the anchor 15 is in the delivery configuration.

The anchor 15 may comprise a series of units 229 connected in series by a wire held to an inner face of each unit 229. The units 229 may abut one another in such a way that when the anchor 15 is in the deployed configuration, the wire length and contact of angled edges between units 229 are configured to provide flexibility/compliance (i.e. degrees of freedom) and allow the anchor 15 to wrap into a screw-like delivery configurations, with a small outer radius, or open into a flat-spiral shaped deployed configuration with a larger radius. The delivery configuration may have a minimum outer diameter 230 and the deployed configuration may have a maximum outer diameter. The wire 20 can bulge through the tie band wire relief 186 when the anchor 15 is in the delivery configuration. The angled edges 184 between units 229 and wire length may be configured such that, when the edges of the units 229 contact one another upon being deployed into the deployed configuration, the outer radius of the anchor 15 is prevented by the wire and the alignment of the units 229 from expanding beyond the maximum outer radius of the deployed configuration.

The anchor 15 may comprise one or more tie band wire reliefs 186 in each unit 229 along an inner surface of the anchor 15. The tie band wire slots 183 may extend along an inner surface of the anchor 15 and provide a gap exposing an inner portion of each unit 229 to allow the tie band wire to bulge through the tie band wire slot 183 when the anchor 15 is in a spiral delivery configuration with a minimum outer radius.

The tie band wire reliefs 186 may further comprise a lipped edge 218. The lipped edge 218 can guide a lipped edge 218 of an adjacent unit 229 such that the movement of the units 229 are restricted by the interface of the lipped edges 218 when the anchor 15 is wrapped around a delivery drive 188 into a compact screw-like spiral shape with a minimum diameter 230. The movement of the units 229 may also be restricted by the interface of the lipped edges when the anchor 15 is released from the delivery drive 188 into a deployed configuration. The lipped edges 218 may line up in the delivery configuration to confine the wire 20 between them.

The anchor 15 may comprise a plurality of intermediate deployed configurations (e.g., as shown in FIGS. 15-17). For example, the anchor 15 may be progressively deployed from the initial delivery configuration to a final deployed configuration though one or more intermediate deployed configurations (e.g., as shown in FIGS. 16-17) in which at least a portion (e.g., a distal portion) of the anchor 15 has a flat spiral shape while another portion (e.g., a proximal portion) remains wound around the delivery drive 188 in a compact screw-like spiral shape. As the anchor 15 continues to deploy towards the final deployed configuration, more and more of the anchor 15 unwinds from the compact screw-like spiral shape into the flat spiral shape until, finally, the entire anchor 15 is deployed.

In some embodiments, the tie band wire may run parallel to the inner circumference of the anchor 15. In some embodiments, the tie band wire may run at an angle to the inner circumference of the anchor 15, for example, in order to help the anchor 15 assume a screw-like helical formation in the delivery configuration.

The tension provided by the tie band wire may be related to the stiffness of the anchor body material and/or the thickness of the tie band wire, as will be understood by one of ordinary skill in the art based on the teachings herein.

The tension provided by the tie band wire may be related to the stiffness of the anchor body material and/or the thickness of the tie band, as will be understood by one or ordinary skill in the art based on the teachings herein. In some embodiments the tie band wire material may comprise biocompatible metals or biocompatible polymers. In some embodiments, the biocompatible polymers can comprise polyethylenterephthalate (PET), polytetrafluoreoethylene (PTFE), polyethylene (PE), etc. In some embodiments, the biocompatible metals can comprise nitinol, titanium, stainless steel, etc.

The anchor 15 may comprise one or more loops as described herein. In some embodiments, the anchor 15 may comprise a single loop. In some embodiments, the anchor 15 may comprise two loops. In some embodiments, the anchor 15 may comprise more than 2 loops. In some embodiments, the anchor 15 may comprise less than one loop (e.g., the anchor 15 may comprise a 270 degree or 315 degree spiral). In some embodiments, the anchor 15 may comprise more than 1 and less than 2 loops (e.g., the anchor 15 may comprise 360 degrees, 385 degrees, 405 degrees, 440 degrees, 450 degrees, 495 degrees, 540 degrees, 585 degrees, 630 degrees, or 675 degrees).

In some embodiments, the anchor 15 may be configured to be locked in the deployed configuration. For example, one or more locking mechanisms may be disposed on the anchor 15 to lock the anchor 15 into place as described herein. In some instances, the locking mechanisms may be disposed on the two ends of the anchor 15 and configured to engage one another (e.g., as a lock and key, etc.) to lock the anchor 15 in the deployed configuration, thereby increasing the compressive stiffness and expansive stiffness of the anchor 15 in the deployment configuration compared to when the two ends are not locked in the deployment configuration. It will be understood by one of ordinary skill in the art that any locking mechanism(s) described herein or known to one of ordinary skill in the art based on teachings herein may be used as desired.

FIGS. 19A-19C show close views of a unit 229 (also referred to herein as a link) of an anchor 15 with pins 190, each unit 229 comprising a tie band retaining pin 190 and tie band retaining tabs 191. FIG. 19A shows a top perspective view. FIG. 19B shows a side plan view. FIG. 19B shows a side view. FIGS. 20A-20B show close perspective and side views, respectively, of a tie band comprising tie band retaining pin constraints 193 and fold relief slots 194 patterned along the length of the tie band.

As shown in FIGS. 19A-19C and 20A-20B the anchor 15 can comprise a series of units 229 connected by a tie band 195. Each unit 229 can comprise tie band retaining tabs 191 and a tie band retaining pin 190. The tie band can comprise tie band retaining pin constraint holes 193 to accommodate tie band retaining pins 190, fold relief slots 194, and a spine 175. The fold relief slots 194 are cut so as to control the flexibility and bend of the tie band 195. The fold relief slots 194 may be cut at an angle of 90 degrees to a spine 175 of the tie band 195. The fold relief slots 194 may be cut at an angle less than 90 degrees to a spine 175 of the tie band 195.

The anchor 15 may comprise a series of units 229 connected in series by a tie band 195 threaded along an inner face of each unit 229. The units 229 may abut one another in such a way that when the anchor 15 is in the deployed configuration, the wire 20 length and angle of contact 184 between units 229 are configured to provide flexibility/compliance (i.e. degrees of freedom) and allow the anchor 15 to wrap into a screw-like delivery configurations, with a small outer radius, or open into a flat-spiral shaped deployed configuration with a larger radius. The delivery configuration may have a minimum outer radius and the deployed configuration may have a maximum outer radius. Fold relief slots 194 cut into the tie band 195 can be cut to control the rigidity of the tie band 195. The fold relief slots 194 can allow for flexibility in the tie band 195 for the anchor 15 to be wrapped into a delivery configuration spiral with a minimum diameter. The angle of contact 184 between units 229 and/or tie band 195 rigidity may be configured such that, when the edges 184 of the units 229 contact one another upon being deployed into the deployed configuration, the outer radius of the anchor 15 is prevented by the wire 20 and the alignment of the units 229 from expanding beyond the maximum outer radius of the deployed configuration.

The anchor 15 may comprise one or more fold relief slots 194 in the tie band 195 held in place in each unit 229 along an inner surface of the anchor 15 by tie band retaining tabs 191. The tie band retaining tabs 191 may extend along an inner surface of each unit 229 of the anchor 15. The anchor 15 may comprise one or more tie band retaining pins 190 which can be slid into tie band retaining pin constraint holes 193 along the tie band 195. The tie band retaining pins 190 may also be configured to act as unit drive pins (similar to unit drive pins 185 described herein) to fit within a groove of a delivery pin guide 188 when the anchor 15 is in the delivery configuration

In some embodiments, the tie band 195 may run parallel to the inner circumference of the anchor 15. In some embodiments, the tie band 195 may run at an angle to the inner circumference of the anchor 15, for example, in order to help the anchor 15 assume a screw-like helical formation in the delivery configuration.

The tension provided by the tie band 195 may be related to the stiffness of the anchor body material and/or the thickness of the tie band 195, as will be understood by one of ordinary skill in the art based on the teachings herein. In some embodiments, the anchor body material may comprise materials with a flexural modulus of less than about 1 gigapascal (GPa), 5 GPa, 10 GPa, 15 GPa, 20 GPa, 25 GPa, 50 GPa, 100 GPa, or 150 GPa. The anchor body material may comprise materials with a flexural modulus of greater than about 1 GPa, 5 GPa, 10 GPa, 15 GPa, 20 GPa, 25 GPa, 50 GPa, 100 GPa, or 150 GPa.

The tension provided by the tie band 195 may be related to the stiffness of the anchor body material, the thickness of the tie band, and/or number and direction of fold relief slots 194, as will be understood by one or ordinary skill in the art based on the teachings herein. The direction of the fold relief slots 194 can aid in guiding the units from a delivery configuration to a rigid deployed configuration. In some embodiments the tie band material may comprise biocompatible metals or biocompatible polymers. In some embodiments, the biocompatible polymers can comprise polyethylenterephthalate (PET), polytetrafluoreoethylene (PTFE), polyethylene (PE), etc. In some embodiments, the biocompatible metals can comprise nitinol, titanium, stainless steel, etc.

The anchor 15 may be configured such that when it is compressed by radial constriction (e.g., by an outer shaft or inner shaft of a delivery device as described herein to retain its delivery configuration) it remains relatively compliant and when it is uncompressed (e.g., in the deployed configuration) it is relatively rigid. The rigidity of the anchor 15 in the deployed configuration may enable the anchor 15 to retain a frame structure as described herein.

The anchor 15 may comprise one or more loops as described herein. In some embodiments, the anchor 15 may comprise a single loop. In some embodiments, the anchor 15 may comprise two loops. In some embodiments, the anchor 15 may comprise more than 2 loops. In some embodiments, the anchor 15 may comprise less than one loop (e.g., the anchor 15 may comprise a 270 degree or 315 degree spiral). In some embodiments, the anchor 15 may comprise more than 1 and less than 2 loops (e.g., the anchor 15 may comprise 360 degrees, 385 degrees, 405 degrees, 440 degrees, 450 degrees, 495 degrees, 540 degrees, 585 degrees, 630 degrees, or 675 degrees).

In some embodiments, the anchor 15 may be configured to be locked in the deployed configuration. For example, one or more locking mechanisms may be disposed on the anchor 15 to lock the anchor 15 into place as described herein. In some instances, the locking mechanisms may be disposed on the two ends of the anchor 15 and configured to engage one another (e.g., as a lock and key, etc.) to lock the anchor 15 in the deployed configuration. The joining of the two ends can increase the compressive stiffness and expansive stiffness of the anchor 15 in the deployment configuration compared to when the two ends are not joined. It will be understood by one of ordinary skill in the art that any locking mechanism(s) described herein or known to one of ordinary skill in the art based on teachings herein may be used as desired.

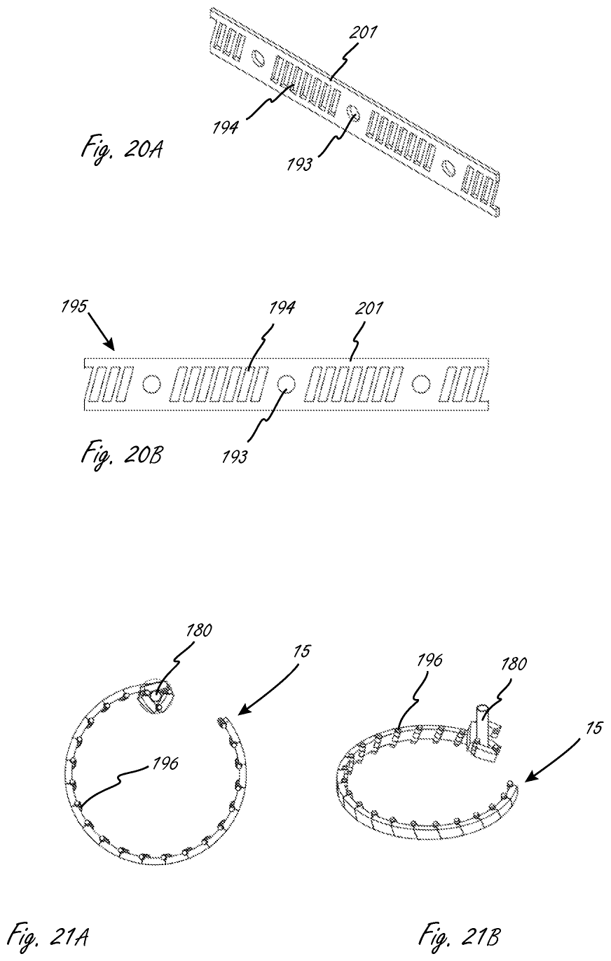

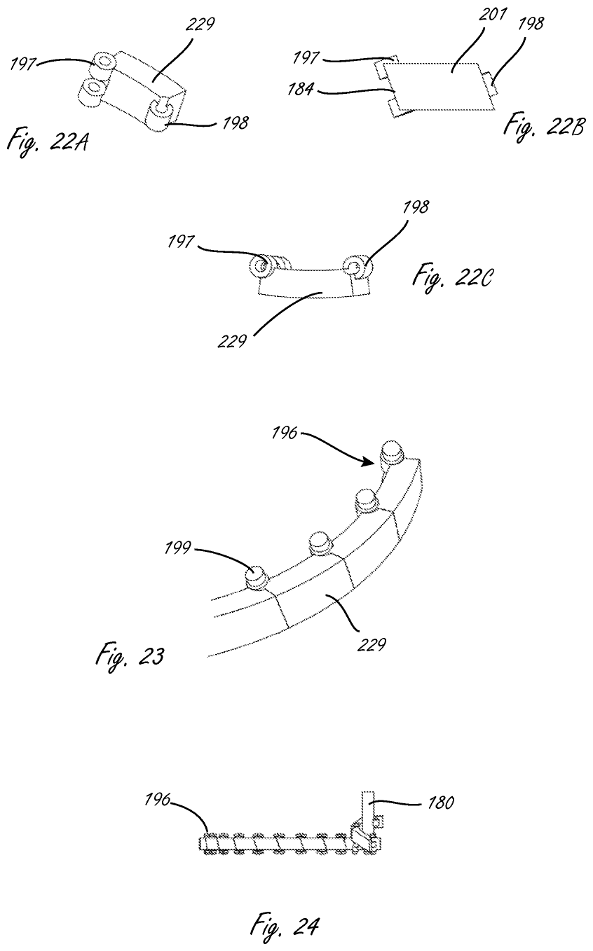

FIGS. 21A-21B shows top and perspective views, respectively, of an anchor 15 comprising a plurality of links 229 coupled together with a plurality of knuckles 196, the anchor 15 being deployed from a delivery configuration disposed around a delivery drive 180 to a deployed configuration. FIGS. 22A-22C show close views of a unit 229 of the anchor 15 of FIGS. 21A-21B, each unit 229 comprising a knuckle element 197 configured to couple to a knuckle element 198 of an adjacent unit with a pin 199 therebetween. FIG. 22A shows a top perspective view. FIG. 22B shows a top view. FIG. 22C shows a side view. FIG. 23 shows a close perspective view of the anchor 15 of FIGS. 21A-21B with hinge pins 199 located between units 229 and holding each unit 229 pair together at their knuckle elements 197, 198. FIG. 24 shows a side view of the anchor 15 of FIGS. 21A-21B comprising knuckles 196 being deployed from the delivery configuration around the delivery drive 180 to the deployed configuration.

As shown in FIGS. 21A, 21B, 22A, 22B, 22C, 23 and 24, the anchor 15 can comprise a series of units 229 connected by knuckle elements 197, 198 which can be held together by a hinge pin 199, or alternatively, a continuous wire stitched from knuckle to knuckle to produce a whole knuckle 196. As is shown in FIG. 22B, the side edges of each unit 229 can comprise an edge/fold angle 184 to facilitate the folding of the anchor 15 into a delivery configuration of a tight spiral, as can be seen in FIGS. 21B and 24. The folding of the anchor 15 into a delivery configuration can be facilitated by the attachment of the anchor 15 to a delivery drive 180, as can be seen in FIGS. 21A, 21B, and 24.

The anchor 15 may comprise a plurality of intermediate deployed configurations (e.g., as shown in FIGS. 21A-21B). For example, the anchor 15 may be progressively deployed from the initial delivery configuration to a final deployed configuration though one or more intermediate deployed configurations (e.g., as shown in FIGS. 21B and 24) in which at least a portion (e.g., a distal portion) of the anchor 15 has a flat spiral shape while another portion (e.g., a proximal portion) remains wound around the delivery drive 180 in a compact screw-like spiral shape. As the anchor 15 continues to deploy towards the final deployed configuration, more and more of the anchor 15 unwinds from the compact screw-like spiral shape into the flat spiral shape until, finally, the entire anchor 15 is deployed.

The anchor 15 may comprise a series of units 229 connected in series by knuckle elements 197, 198 held together by a hinge pin 199 or wire disposed within each knuckle element 196. The units 229 may abut one another in such a way that when the anchor 15 is in the deployed configuration, the knuckle alignment and angle of contact between units 229 are configured to provide flexibility/compliance (i.e. degrees of freedom) and allow the anchor 15 to wrap into a screw-like delivery configurations, with a small outer radius, or open into a flat-spiral shaped deployed configuration with a larger radius. The delivery configuration may have a minimum outer radius and the deployed configuration may have a maximum outer radius. The angled edges 184 between units 229 and knuckle element 198, 197 alignment may be configured such that, when the edges 184 of the units 229 contact one another upon being deployed into the deployed configuration, the outer radius of the anchor 15 is prevented by the knuckle 198, 197 alignment and the alignment of the edges 184 of the abutting units 229 from expanding beyond the maximum outer radius of the deployed configuration.

Alternatively, a wire can be threaded through a lumen of each knuckle element 197, 198 to connect the knuckles. The wire may be threaded in a S-pattern along an inside edge of the units 229 and through the knuckle elements 197, 198 of the units 229.

The tension provided by the knuckle 196 may be related to the contact angle 184 of each unit and the radial constriction on the pins 199 by the hinge elements 197, 198.

The tension provided by the tie band wire may be related to the stiffness of the anchor body material and/or the thickness of the tie band, as will be understood by one or ordinary skill in the art based on the teachings herein. In some embodiments the tie band wire material may comprise biocompatible metals or biocompatible polymers. In some embodiments, the biocompatible polymers can comprise polyethylenterephthalate (PET), polytetrafluoreoethylene (PTFE), polyethylene (PE), etc. In some embodiments, the biocompatible metals can comprise nitinol, titanium, stainless steel, etc.

The anchor 15 may comprise a plurality of intermediate deployed configurations (e.g., as shown in FIGS. 21A-21B). For example, the anchor 15 may be progressively deployed from the initial delivery configuration to a final deployed configuration through one or more intermediate deployed configurations (e.g., as shown in FIGS. 21A-21B) in which at least a portion (e.g., a distal portion) of the anchor 15 has a flat spiral shape while another portion (e.g., a proximal portion) remains wound around the delivery drive 180 in a compact screw-like spiral shape. As the anchor 15 continues to deploy towards the final deployed configuration, more and more of the anchor 15 unwinds from the compact screw-like spiral shape into the flat are, circular, or spiral shape until, finally, the entire anchor 15 is deployed.

The tension provided by anchor may be related to the stiffness of the anchor body material and/or the thickness of the anchor, as will be understood by one of ordinary skill in the art based on the teachings herein. In some embodiments the anchor may comprise biocompatible metals or biocompatible polymers. In some embodiments, the biocompatible polymers can comprise polyethylenterephthalate (PET), polytetrafluoreoethylene (PTFE), polyethylene (PE), etc. In some embodiments, the biocompatible metals can comprise nitinol, titanium, stainless steel, etc.

The anchor 15 may comprise one or more loops as described herein. In some embodiments, the anchor 15 may comprise a single loop. In some embodiments, the anchor 15 may comprise two loops. In some embodiments, the anchor 15 may comprise more than 2 loops. In some embodiments, the anchor 15 may comprise less than one loop (e.g., the anchor 15 may comprise a 270 degree or 315 degree spiral). In some embodiments, the anchor 15 may comprise more than 1 and less than 2 loops (e.g., the anchor 15 may comprise 360 degrees, 385 degrees, 405 degrees, 440 degrees, 450 degrees, 495 degrees, 540 degrees, 585 degrees, 630 degrees, or 675 degrees).

In some embodiments, the anchor 15 may be configured to be locked in the deployed configuration. For example, one or more locking mechanisms may be disposed on the anchor 15 to lock the anchor 15 into place as described herein. In some instances, the locking mechanisms may be disposed on the two ends of the anchor 15 and configured to engage one another (e.g., as a lock and key, etc.) to lock the anchor 15 in the deployed configuration. It will be understood by one of ordinary skill in the art that any locking mechanism(s) described herein or known to one of ordinary skill in the art based on teachings herein may be used as desired.

In some embodiments, the anchor 15 may comprise a super-elastic material. In some embodiments, the anchor 15 may comprise nitinol. In some embodiments, the anchor 15 may comprise one or more channels or lumens disposed therein. The anchor 15 body may comprise a hollow, tubular cross-section. The anchor 15 may, for example, comprise a hypotube. The lumen of the anchor 15 may be configured to pass another component (e.g., a tie band or wire as described herein) therethrough. The anchor 15 may comprise one or more relief cuts (e.g., laser-cuts or the like) in an outer perimeter of the anchor body 15 extending into the one or more lumens in order to provide flexibility to the anchor 15 and enable the anchor to coil into a coiled screw-like delivery configuration as described herein.

The tie band 195 or tie band wire may be formed of a material having sufficient rigidity to hold a predetermined shape. The tie band may, for example, be formed of a shape memory material (e.g., NiTi). It may be desirable for at least an end portion (e.g., free end 22) to be relatively rigid such that it can exert a force to move the leaflets and/or chordal tendineae, while still retaining flexibility to be collapsed within a delivery device. In various embodiments, the end portion only needs sufficient rigidity to hold its shape and will deform under a load. For example, the end portion may be configured with a similar rigidity to a guidewire, or slightly stiffer.

In some embodiments, the tie band 195 or tie band wire may comprise a super-elastic material. In some embodiments, the tie band or tie band wire may comprise nitinol. In some embodiments, the tie band may comprise a nitinol wire.

In some embodiments the anchor 15 may comprise biocompatible metals or biocompatible polymers. In some embodiments, the biocompatible polymers can comprise polyethylenterephthalate (PET), polytetrafluoreoethylene (PTFE), polyethylene (PE), etc. In some embodiments, the biocompatible metals can comprise nitinol, titanium, stainless steel, etc.

As described herein, each unit 229 (also referred to herein as a link) of the anchor 15 can have a slanted geometry so that when the units 229 are linked together, the multi-linked anchor 15 can transition from an elongated helical structure while in a delivery configuration to a flat structure (e.g., flat arc, circle, or spiral) when in a deployed configuration. As describe above, the units 229 may be coupled together by a tie band (e.g. wire) or may be coupled together by pins. The anchor 15 can include an expansion spring that has an arc, circular, or spiral shape in accordance with the flat arc, circular, or spiral shape of the anchor when in the expanded deployed state. In cases where the anchor includes a tie band, the tie band can correspond to the expansion spring. In cases where the anchor 15 includes pins to hold the units 229 together, the expansion spring may run through the units 229 or be positioned adjacent to the units 229.

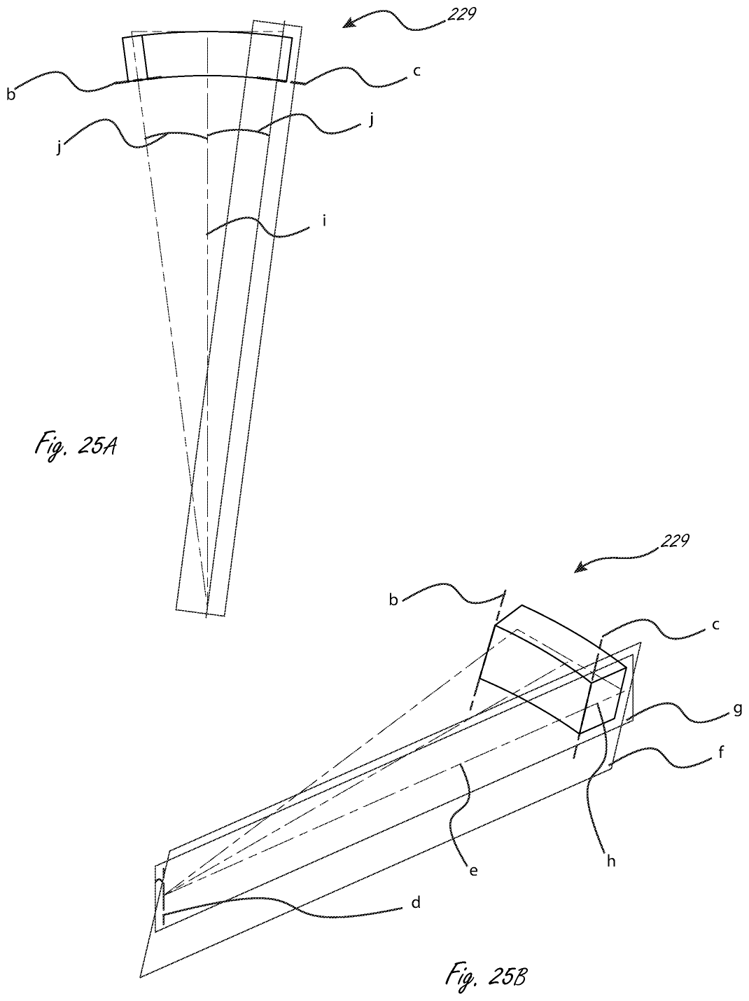

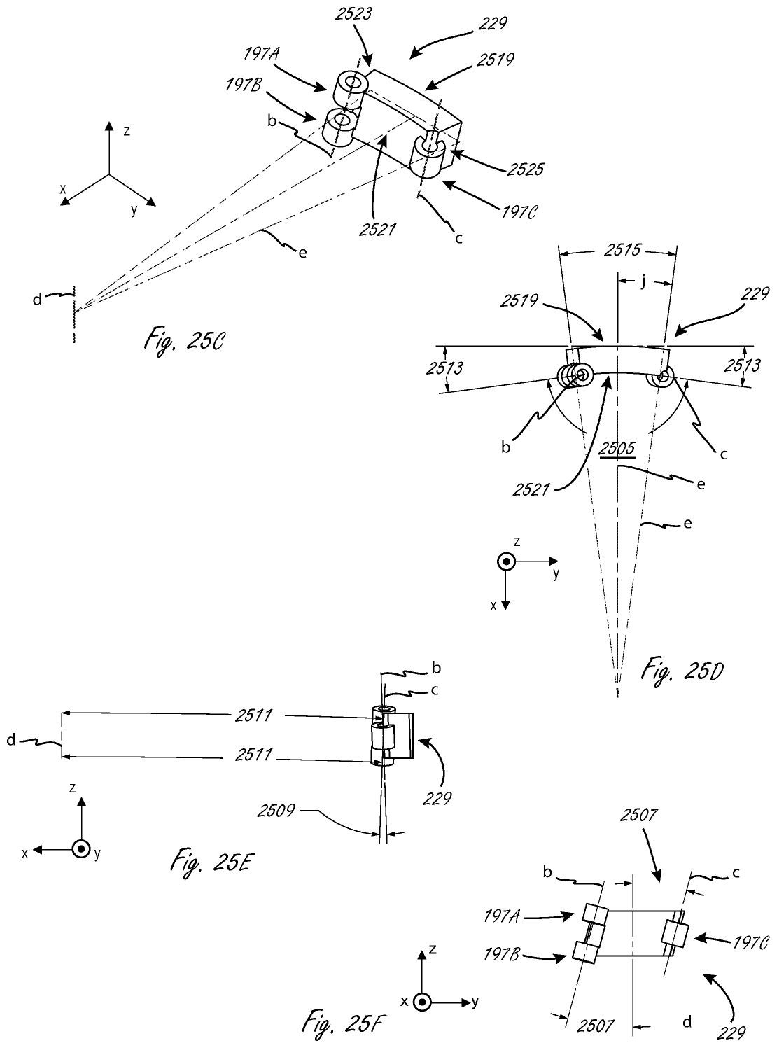

FIGS. 25A and 25B show aspects of a unit 229, which includes a midline h, a centerline i, and a half angle j. An intersection line e is at the intersection of an axial reference plane g and a cut plane f. An anchor central axis d defines the central axis of the anchor when deployed in the flat shape (e.g., flat arc, circle, or spiral). Axes b and c are rotation axes about which knuckle elements can be rotated (see FIG. 25C). A rotation axis (e.g., c) can be located within a cut plane of a corresponding face (e.g., f). Each segment has two faces, each with a corresponding cut plane (e.g., f and ff, not shown).

FIGS. 25C-25F show the unit 229 with three knuckle elements 197A, 197B, and 197C that protrude from an inner side 2521 of the unit 229. The curved outer side 2519 corresponds to a side of the unit 229 that forms the outer perimeter of the flat shape (e.g., flat arc, circle, or spiral) when the anchor is deployed. The curved inner side 2519 of the unit 229 is opposite the outer side 2519 and corresponds to the inner surface of the flat shape (e.g., flat arc, circle, or spiral) when the anchor is deployed. The first and second knuckle elements 197A, 197B are positioned along an inner edge of a first radial side 2523 of the unit 229 and comprise through-features (e.g., holes) that define an axis of rotation b. The first and second knuckle elements 197A and 197B are spaced apart such that a single knuckle element of an adjacent unit (not shown) can fit therebetween. A third knuckle element 197C is along a second radial side 2525 of the unit 229 and comprises a through-feature that defines an axis of rotation c. The third knuckle element 197C is configured to fit between two knuckle elements of another adjacent unit (not shown). When the anchor is fully deployed, the first radial side 2523 and the second radial side 2525 contact the adjacent corresponding units so that the flat arc, circle or spiral shape is fully expanded and held stiffly in place by securing elements (e.g., pins).

During deployment of the anchor, the unit 229 is configured to rotate about the (e.g., first) rotational axis b of the first and second knuckle element 197A, 197B and the (e.g., second) rotational axis c of the third knuckle element 197C. The first knuckle element 197A and the second knuckle element 197B are arranged such that the first rotational axis b is coaxial with the inside edge of the first radial side 2523 of the unit 229. The third knuckle element 197C is arranged such that the second rotational axis c is coaxial with the inside edge of the second radial side 2525 of the unit 229. The first rotational axis b and the second rotational axis c are parallel to each other, and are each laterally offset with respect to the anchor central axis d by an edge angle 2507 in a projection plane parallel to the central axis d containing points of intersection of a radius e and the axis b and a point of intersection of another radius e and the axis c (e.g., projection plane y-z, FIG. 25F). The edge angle 2507 corresponds to the angle at which the sides 2523 and 2525 of the unit 229 are skewed and that allow the anchor 15 to wind into a helical shape. In some cases, the edge angle 2507 between the first rotational axis b and the second rotational axis c can correspond to the delivery pitch angle for coiling the anchor around the delivery gear. In some cases, the edge angle 2507 can be within a range bounded by any two of the following values: 5 degrees, 10 degrees, 15 degrees, 20 degrees, 25 degrees, 30 degrees, 35 degrees, 40 degrees, 45 degrees, 50 degrees, 55 degrees, and 60 degrees.

The knuckle elements 197A, 197B, and 197C are arranged so that the rotational axes b and c are rotationally offset with respect to each other so that they do not exist along the same plane. The offset rotational axes configuration, along with the slanted geometry of the unit 229, allows the anchor to transition between the elongated helical structure (delivery configuration) to the flat structure (deployed configuration). For example, FIG. 25E shows that the first rotational axis b is offset (non-parallel to) with respect to the second rotational axis c by an angle 2509 in a projection plane containing the central radius e and the central axis (e.g., projection plane x-z, FIG. 25E). In some cases, the angle 2509 can be within a range bounded by any of the two of the following values: 1 degree, 2 degrees, 3 degrees, 4 degrees, 5 degrees, 6 degrees, 7 degrees, 8 degrees, 9 degrees, and 10 degrees. Further, none of the rotational axes of the multiple units of the anchor exist along the same plane. That is, each of the units within the anchor can include two rotational axes that are rotationally offset with respect to each other and with respect to other rotational axes of other units in the anchor. In addition, the first rotational axis b and the second rotation axis c can be offset (non-parallel to) with respect to the anchor central axis d by an angle 2511 projected onto the plane containing the central radius e and the central axis (e.g., projection plane X-z, FIG. 25E). In some cases, the angle 2511 can be within a range bounded by any of the two of the following values: 1 degree, 2 degrees, 3 degrees, 4 degrees, 5 degrees, 6 degrees, 7 degrees, 8 degrees, 9 degrees, and 10 degrees. In some cases, the angle 2509 is greater than the angle 2511.

FIG. 25D show other dimensions of the unit 229 as projected in a plane containing a radius e and normal to the central axis d. An angle 2515, which is twice the half angle j, can be within a range bounded by any of the two of the following values: 5 degrees, 10 degrees, 15 degrees, 20 degrees, and 25 degrees. An angle 2513 from a line tangent to the outer-most point on the outer side 2519 to an inner-most edge of the first radial side 2523 or second radial side 2525 can be within a range bounded by any of the two of the following values: 5 degrees, 10 degrees, 15 degrees, and 20 degrees. An angle 2505 between the inner-most edge of the first radial side 2523 and the inner-most edge of the second radial side 2525 can be within a range bounded by any of the two of the following values: 150 degrees, 160 degrees, 165 degrees, 170 degrees, and 180 degrees.

Methods of Use

The distal end of the delivery device 30 may be configured to be advanced from a first side of a native valve to a second side of the native valve. For example, the distal end of the delivery device 30 may be advanced from a left atrial side of a mitral valve to a left ventricular side of a mitral valve. In some instances, the distal end of the delivery device 30 may be transseptally inserted into the left atrium of the heart prior to advancement into the left ventricle. Alternatively, or in combination, the distal end of the delivery device 30 may be steerable such that it is positionable to point towards the first side of the native valve before being advanced to the second side of the native valve.

After advancing the distal end of the delivery device to the second side of the native valve, the anchor 15 can be unwound from the elongated delivery state to the flat deployed state on the second side of the native valve. During deployment, some of the anchor 15 may be in the elongated delivery state on first side of the native and some of the anchor 15 may be in the flat deployed state on the second side of the native valve until the anchor is fully unwound and deployed. The anchor 15 may be deployed in the deployed state just past the native valve and as close to the native valve annulus as possible. In alternate embodiments it may be distanced from the annulus. In some embodiments, fully deploying the anchor 15 may comprise positioning the anchor 15 such that it is located only on the second side of the native valve.

In some cases, after advancing the distal end of the delivery device to the second side of the native valve, the anchor 15 can be unwound from the elongated delivery state to a spiral deployed form, which may include portions, or not includes portions, on the atrial side.

In some cases, the delivery device is configured to deliver the anchor in the wound elongated delivery state completely into the second side (e.g., ventricle), then be delivered from a place proximal to the end of the delivery device out of the side of the delivery device around the annulus. The frame structure of the valve prosthesis can then be delivered into and deployed within the native valve adjacent to the deployed anchor.

Alternatively, the anchor 15 may be deployed on the first side of the valve (e.g., in one of the atria) and then pushed through the respective valve and subsequently rotated to anchor to the chordae and/or native valve.

Advancing the anchor 15 may comprise pushing the anchor 15 through the native valve. Advancing the anchor 15 may further comprise rotating the anchor 15 through the native valve. Advancing the anchor 15 may comprise pushing the anchor out of an inner sheath of a delivery device. Advancing the anchor 15 may comprise pushing the inner sheath of the delivery device through the native valve before pushing the anchor out of the inner sheath.

Advancing the anchor 15 may comprise deploying the anchor 15 from an anchor delivery drive 180, as can be seen in FIGS. 4A-4D and FIGS. 3B-3C. Deploying the anchor 15 may occur simultaneously with securing the anchor 15 around the native chordae tendineae 40 and/or native leaflets 42 of the diseased valve 4, by rotating the anchor 15 off of the delivery drive 180 and/or by releasing the anchor 15 from radial construction and allowing it to unwind off the delivery drive 180. Deployment of the anchor 15 may occur on a ventricle side 26 of the diseased valve 4.

Deployment of the anchor 15 may occur by release from an anchor delivery gear. For example, as shown in FIGS. 6 and 7, the anchor 15 can comprise a laser-cut anchor series of pseudo-units attached to a series of teeth 181 on an anchor delivery gear 180. The anchor delivery gear 180 may be coupled to an inner shaft 52 of the delivery device 30. In some embodiments, a distal position of the inner shaft 52 may comprise the anchor delivery gear 180. In some embodiments, for example as shown in FIGS. 14, 15 and 16, the anchor can comprise a series of interconnected units. Each unit may have a unit drive pin 185 configured to be retained by a delivery pin guide 187 on a delivery drive 188.

In some embodiments, for example as shown in FIGS. 3A-3B the anchor 15 can be delivered in the delivery configuration within an inner shaft 52 of a delivery device 30. The anchor 15 can be deployed from a delivery gear 180 into a deployed configuration out of a distal end of the inner shaft 52. Alternatively, the anchor 15 can be deployed from a side port of the inner shaft 52 or a lateral opening formed by retraction of the outer sheath 50.

In some embodiments, the anchor 15 may be actuated from the delivery configuration to the deployed configuration on a first side of the native valve prior to being advanced to a second side of the native valve. For example, the anchor 15 may be deployed in a left atrium of a heart prior to being advanced to a left ventricle of the heart as described herein.

Alternatively, the anchor 15 may be actuated from the delivery configuration to the deployed configuration on a second side of the native valve after being advanced to the second side from a first side of the native valve. For example, anchor 15 may be advanced from a left atrium of a heart prior to being deployed in a left ventricle of the heart by the retreat of an outer sheath or advancement out of an inner shaft 52.

The free end 22 of the deployed anchor 15 may optionally be rotated around one or more structures on the second side of the native valve. The one or more structures may comprise one or more valve leaflets of the native valve. Alternatively, or in combination, the one or more structures may comprise one or more chordae of the left ventricle.

The free end 22 of the deployed anchor 15 may optionally rotated around one or more structures on the second side of the native valve such that the one or more structures (e.g., chordae, leaflets, or annulus) are pulled radially inwards towards the longitudinal axis of the anchor 15m and/or towards the longitudinal axis of the delivery device 30. The anchor 15 and/or free end 22 may be configured such that minimal torque is applied to the one or more structures. Alternatively, or in combination, the anchor 15 and/or free end 22 may be configured such that the one or more structures are not rotated, or are minimally rotated, during rotation of the anchor 15.

The anchor 15 may then be released from the distal end of the delivery device 30. The anchor 15 may be released from the distal end of the delivery device 30 on the second side of the native valve.

The frame structure 12 may be expanded within the native valve from an unexpanded configuration to an expanded configuration.

The frame structure 12 may be released from the distal end of the delivery device 30. In some embodiments, at least a portion the frame structure 12 may be expanded within at least a portion of the deployed anchor to anchor the frame structure 12 to the native valve.

In some embodiments, expanding the frame structure 12 and releasing the frame structure 12 may occur simultaneously.

Finally, the delivery device 30 may be retracted from the native valve.

FIGS. 2A, 2B and 3F show the valve prosthesis 10 fully expanded with the native valve leaflets 42 and chordae tendineae 40 captured between the frame structure 12 and the anchor 15.

Although the steps above show a method of deploying a valve prosthesis 10 within a native valve 4 in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as necessary to assemble at least a part of an article.

For example, in some embodiments deploying the valve prosthesis 10 may occur in multiple steps such that a portion of the valve prosthesis 10 (e.g., anchor 15) may be deployed before another portion the valve prosthesis 10 (e.g., frame structure 12). Alternatively, or in combination, in some embodiments, deploying the anchor 15 may occur in multiple steps such that a portion of the anchor 15 may be deployed before being advanced through the native valve 4 and another portion of the anchor 15 may be deployed after being advanced through the native valve 4. Alternatively, or in combination, the delivery device 30 may be advanced from the left atrium 25 to the left ventricle 26 with the valve prosthesis 10 undeployed. In many embodiments, the frame structure 12 may be balloon-expandable and the delivery device 30 may comprise a balloon in order to expand the frame structure 12. Alternatively, or in combination, the anchor 15 may be released after the frame structure 12 has been expanded within it.

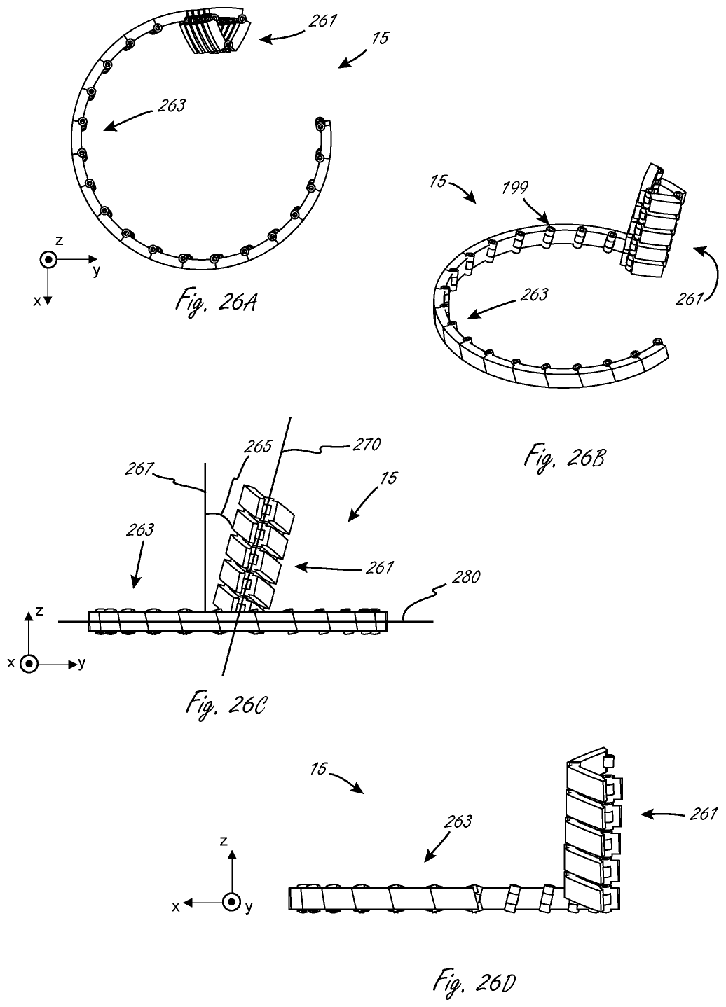

FIGS. 26A-26D show various views of an anchor 15 assembled using multiple units 229 of FIGS. 25A-25F, where the anchor 15 is in a partially deployed state. Note that the anchor 15 in this example includes more unit 229 than required to form a complete circle and is shown for illustrative purposes. During deployment, a first portion 261 of the anchor 15 may have a helical shape around the delivery gear (e.g., 180 in FIGS. 4A-7B) in accordance with the collapsed configuration and a second portion 263 of the anchor 15 may have an expanded (e.g., flat) shape in accordance with the delivery configuration. The second portion 263 of the anchor 15 lies along a deployed plane 280 (e.g., along the xy axis). A deployment axis 270, which is parallel to a central axis of the first portion 261 (undeployed portion) of the anchor 15, is at a deployment angle 265 with respect to a reference axis 267, where the reference axis 267 (e.g., along the xz axis) is normal to the deployed plane 280. The deployment angle 265 can be adjusted to control the angle at which the anchor 15 can be deployed, for example, from a delivery catheter. The deployment angle 265 may correspond to the edge angle 2507 of the sides of the units 229. In some cases, the deployment angle 265 may correspond to the pitch angle 240 described above. The angle deployment angle 265 can be within a range bounded by any two of the following values: 5 degrees, 10 degrees, 15 degrees, 20 degrees, 25 degrees, 30 degrees, 35 degrees, 40 degrees, 45 degrees, 50 degrees, 55 degrees, and 60 degrees.

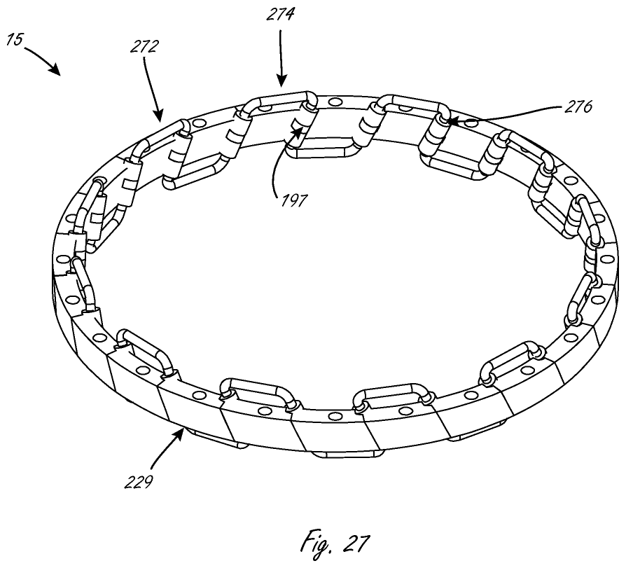

The pseudo units/units of any of the anchors 15 describe herein can be coupled together using any of a number of mechanisms. In some cases, pseudo units are connected by a tie band that corresponds to a material at an inner radius of the anchor, such as illustrated in the examples of FIGS. 5A-10. In some cases, a tie band and/or spring wire positioned adjacent to or runs through individual units and has a circular or arc shape in accordance with a circular or arc shape of the anchor 15, such as illustrated in the examples of FIGS. 11-20B. In other examples, the anchor 15 includes a series of pins that are positioned within knuckles of the units to hold the units together, such as illustrated in the examples of FIGS. 21A-26D. FIG. 27 shows a variation where the anchor 15 includes a tie band 272 that forms a circular or arc shape in accordance with the circular or arc shape of the anchor 15 and that also includes pin portions. In this example, the tie band 272 has a serpentine shape where some portions 274 of the tie band 272 run parallel to the plane of the anchor 15 when in a flat deployed configuration and pin portions 276 are positioned within the knuckles 197, thereby acting as pins that couple the units 229 together.

Any of the anchors 15 described herein can be used in a number of applications. That is, the anchors 15 are not limited for use in the deployment of valve prosthesis. For example, the anchors 15 may be used in conjunction with any of a number of catheter-based systems, including steerable catheter-based systems. For instance, the anchors 15 may be part of, or used with, any of a number of imaging catheter systems, diagnostic catheter systems, delivery catheter systems (i.e. for delivery of a device through the delivery catheter), catheter-based therapeutic device systems, and/or robotic surgery catheter systems. In general, the anchors 15 may be used to secure a catheter, or a device as part of a catheter-based system, to a location in the human anatomy.

Any of the anchor 15 describe herein can be delivered from within a lumen of the body to outside the lumen of the body through an incision. As such, the anchor 15 can be used as a means to anchor valves in many types of lumens in the body. Some exemplary lumens follow: 1) In any body lumen to treat conditions where backflow becomes a problem such as in digestive track to tract such as GERD, or the venous system wherein venous valve require replacement; 2) In any body lumen as a means of holding a stent in place to maintain patency in the lumen threatened by closure from tumors such as in the esophagus, intestines, or trachea. Such an anchoring system would allow for anchoring the device without distending the native lumen; 3) The anchor can be used to encircle the inside of a lumen such as to deliver a filter for use in the circulatory system. Such a device can be used to in a fashion where it anchors itself on the inside of the lumen as in an implantable blood filter. Alternatively, it can remain attached to the delivery tool such as in a blood filter which is placed on the distal side of a clot and then cleaned prior to removal of the tool.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-77. (canceled)

78. A device for treating a diseased valve in a patient, the device comprising:
a valve prothesis comprising a frame structure and an anchor, wherein the anchor comprises
a series of segments operably coupled to one another and having a free end,
wherein the anchor has a delivery configuration and a deployed configuration, wherein the anchor is configured to secure the valve prosthesis to the diseased valve,
wherein the anchor is configured to have a low compressive stiffness and low expansive stiffness in the delivery configuration and a high expansion stiffness after transition to the deployed configuration.

79-82. (canceled)

83. The device of claim 78, wherein at least one edge of the segments is slanted.

84-93. (canceled)

94. The device of claim 78, wherein the anchor is configured to be in the radially collapsed delivery configuration when within a delivery device.

95-102. (canceled)

103. The device of claim 78, wherein the free end is configured to extend radially outward when being deployed.

104-105. (canceled)

106. The device of claim 78, wherein the anchor includes a locking mechanism configured to lock the free end and a second end of the anchor together when in a radially expanded deployed configuration.

107. The device of claim 78, wherein a distal end of the anchor comprises a key configured to slide into a complementary lock located on a band of the component.

108. The device of claim 78, wherein the anchor has a flat shape when in a radially expanded deployed configuration.

109-114. (canceled)

115. The device of claim 78, wherein the anchor is configured to coil around a delivery gear when in a radially collapsed delivery configuration.

116. The device of claim 115, wherein each of the series of segments has angled edges in accordance with a delivery pitch angle for coiling the anchor around the delivery gear such that the anchor has a flat shape when in a radially expanded deployed configuration.

117. The device of claim 116, wherein the delivery pitch angle ranges from 5 degrees and 85 degrees.

118. The device of claim 78, wherein each of the series of segments has angled edges that allow the anchor to take on a helical shape when in a radially collapsed delivery configuration.

119-121. (canceled)

122. The device of claim 78, wherein the segments are pseudo units connected by a material at an inner radius of the anchor.

123. The device of claim 122, wherein the material at the inner radius of the anchor corresponds to a tie band that applies a radial expansion force to maintain the anchor in a radially expanded deployed configuration.

124. The device of claim 78, wherein the segments are units that are single entities coupled together by a coupling structure.

125. The device of claim 124, wherein the coupling structure is a band or wire that applies a radial expansion force to maintain the anchor in a radially expanded deployed configuration.
